SEQUENCE LISTING (18 sequences)

SEQ ID NO: 1 - protein, 364 aa, Neisseria meningitidis
MSMKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 2 - protein, 362 aa, Neisseria meningitidis
MKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 3 - protein, 398 aa, Neisseria meningitidis
MKHFPSKVLTTAILATFCSGALAATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSITEHGTRLNGLDRTVSDLRKETRQGLAEQAALSGLFQPYNVGRFNVTAAVGGYKSESAVAIGTGFRFTENFAAKAGVAVGTSSGSSAAYHVGVNYEW

SEQ ID NO: 4 - protein, 405 aa, Neisseria meningitidis
MKHFPSKVLTTAILATFCSGALAATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVGRFNVTAAVGGYKSESAVAIGTGFRFTENFAAKAGVAVGTSSGSSAAYHVGVNYEW

SEQ ID NO: 5 - protein, 323 aa, Neisseria meningitidis
MKHFPSKVLTAAILAALSGSAMADNAPTADEIAKAALVNSYNNTQDINGFTVGDTIYDIKNDKITKKEATEADVEADDFKGLGLKEVVAQHDQSLADLTETVNENSEALVKTAAVVNDISADVKANTAAIGENKAAIATKADKTELDKVSGKVTENETAIGKKANSADVYTKAEVYTKQESDNRFVKISDGIGNLNTTANGLETRLAAAEQSVADHGTRLASAEKSITEHGTRLNGLDRTVSDLRKETRQGLAEQAALSGLFQPYNVGRFNVTAAVGGYKSESAVAIGTGFRFTENFAAKAGVAVGTSSGSSAAYHVGVNYEW

SEQ ID NO: 6 - DNA, 21 bp, Artificial Sequence
  Primer IL-12p35 sense
atggccctgtgccttagtagt

SEQ ID NO: 7 - DNA, 22 bp, Artificial Sequence
  Primer IL-12p35 antisense
cggttcttcaagggaggatttt

SEQ ID NO: 8 - DNA, 21 bp, Artificial Sequence
  Primer IL-12p40 sense
acaaaggaggcgaggttctaa

SEQ ID NO: 9 - DNA, 18 bp, Artificial Sequence
  Primer IL-12p40 antisense
cccttgggggtcagaaga

SEQ ID NO: 10 - DNA, 20 bp, Artificial Sequence
  Primer IL-23p19 sense
tccaccagggtctgattttt

SEQ ID NO: 11 - DNA, 20 bp, Artificial Sequence
  Primer IL-23p19 antisense
ttgaagcggagaaggagacg

SEQ ID NO: 12 - DNA, 22 bp, Artificial Sequence
  Primer TNF-a sense
atgagcactgaaagcatgatcc

SEQ ID NO: 13 - DNA, 22 bp, Artificial Sequence
  Primer TNF-a antisense
gagggctgattagagagaggtc

SEQ ID NO: 14 - DNA, 22 bp, Artificial Sequence
  Primer IL-6 sense
aacctgaaccttccaaagatgg

SEQ ID NO: 15 - DNA, 21 bp, Artificial Sequence
  Primer IL-6 antisense
tctggcttgttcctcactact

SEQ ID NO: 16 - DNA, 16 bp, Artificial Sequence
  primer HMBS sense
ggcaatgcggctgcaa

SEQ ID NO: 17 - DNA, 19 bp, Artificial Sequence
  Primer HMBS antisense
gggtacccacgcgaatcac

SEQ ID NO: 18 - protein, 350 aa, Artificial Sequence
  NadAdelta351-405
MKHFPSKVLTTAILATFCSGALAATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVG

(19) United States
(12) Patent Application Publication
(10) Pub. No.: US 2011/0008279 A1
(43) Pub. Date: Jan. 13, 2011

(54) Methods and Compositions Relating to Adhesins as Adjuvants

(76) Inventors: Vega Masignani, Emeryville, CA (US); Maria Scarselli, Siena (IT); Rino Rappuoli, Siena (IT); Mariagrazia Pizza, Siena (IT); Marzia Monica Giuliani, Siena (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(21) Appl. No.: 12/086,226

(22) PCT Filed: Dec. 6, 2006
(86) PCT No.: PCT/IB2006/003908

§ 371 Date: Jul. 16, 2009

Related U.S. Application Data
(60) Provisional application No. 60/748,109, filed on Dec. 6, 2005.
(60) Provisional application No. 60/844,444, filed on Sep. 13, 2006.

(51) Int. Cl. A61K 38/20; A61K 38/21; A61K 39/39; A61P 37/04

(52) U.S. Cl. 424/85.2; 424/85.4; 424/85.5; 424/184.1; 424/234.1

(57) ABSTRACT

This invention is in the field of immunology and relates to the discovery that adhesins are potent activators of dendritic cells.

### RELATED APPLICATIONS

This application is the U.S. National Phase of International Application No. PCT/IB2006/003908, filed Dec. 6, 2006 and published in English, which claims priority to U.S. Provisional application 60/748,109 filed Dec. 6, 2005 and U.S. Provisional application 60/844,444 filed Sep. 13, 2006. The teachings of the above applications are incorporated in their entirety by reference.

All documents cited herein are incorporated by reference in their entirety.

### TECHNICAL FIELD

This invention is in the field of immunology and relates to the discovery that adhesins are potent activators of dendritic cells.

### BACKGROUND ART

Dendritic cells (DCs) are the antigen presenting cells essential to initiate primary immune response. Present in several tissue, they capture antigens and, matured by typical microbial molecules, or Pathogen Associated Microbial Patterns (PAMPs), migrate to the closest lymphoid tissue where they present antigens to T lymphocytes, which proliferate, differentiate and begin the immune response. Differentiation of naive CD4⁺ T lymphocytes into effector cells producing a selective patterns of cytokines has a deep influence on the kind of immune response which is set up: IFN-γ, produced by Th1 cells, favours cell-mediated immunity and the production of opsonizing and complement-fixing antibodies, while IL-4 produced by Th2 cells promote humoral immunity with the production of neutralising antibodies and defence against elmintic infection [1, 2]. Differentiation of naive T cells mostly results from the cytokine milieu generated by activated DCs, with IL-12 acting as the most powerful Th1-promoting factor. In addition, other factors, including the degree of DC maturation and the expression of costimulatory molecules, determine the pattern of cytokine produced by the differentiated Th cells. DC differentiation signals are determined by a co-stimulation due to microbial factors and to mediators released by other immune and inflammatory cells. One of the most powerful DC potentiating agents is IFN-γ, a cytokine mostly produced by NK and by Th1 memory cells, Priming with IFN-γ strongly increases LPS-induced production of IL-12.

Some tumours are able to produce a number of immunosuppressive factors that block the maturation of DCs from CD34⁺ cells or CD14⁺ blood monocytes. Thus, providing mature, activated DCs to a subject overcomes this issue. However, the DCs must first be activated in vitro.

Endotoxin (LPS) is a major stimulus converting immature DCs into fully functional APC, which secrete large amounts of soluble mediators like chemokines and cytokines. T lymphocytes activated by LPS-treated DCs strongly polarise toward the IFN-γ-producing Th1 phenotype, which favours the inflammatory response and cell-dependent immunity.

Thus there is a need to find other stimuli for converting immature DCs into fully functional APCs. There is also a need to find new adjuvants for use with vaccination.

### SUMMARY OF THE INVENTION

In one aspect, the present invention provides methods of adjuvanting an immune response, comprising administering an effective amount of a composition comprising an adhesin. In one embodiment, dendritic cells are activated by administering an effective amount of a composition comprising an adhesin. In a particular embodiment, the adhesin comprises a soluble form of NadA. In a further embodiment, the composition further comprises an additional adjuvant and/or immunopotentiator. In a particular embodiment, the additional adjuvant and/or immunopotentiator is selected from an immunostimulatory oligonucleotide, an oil-in-water emulsion, a mineral salt, an ISCOM, LPS or an imidazoquinoline compound.

In another aspect, the present invention provides compositions comprising an adhesin, an antigen and one or more of an immunostimulatory oligonucleotide, an oil-in-water emulsion, a mineral salt, an ISCOM, LPS or an imidazoquinoline compound. In one embodiment, the adhesin is a soluble form of NadA. In a further embodiment, the soluble form of NadA is NadAΔ351-405.

The methods and compositions of the invention may further comprise an interleukin or an interferon. In a particular embodiment, the interferon is IFN-γ.

In a further aspect, the present invention provides for use of a composition of the invention for adjuvanting an immune response. In another aspect, the present invention also provides for use of compositions of the invention for activating and sensitising a dendritic cell. In a particular embodiment, the dendritic cell is CD86⁻.

### DISCLOSURE OF THE INVENTION

It has been discovered that NadA binds to monocyte derived dendritic cells and, when they are primed with IFN-γ, activates them. Therefore, NadA and other adhesins, e.g., other bacterial adhesins, preferably bacterial epithelial adhesins, may be used to activate dendritic cells and/or act as immuopotentiators.

The invention therefore provides a method of activating dendritic cells, comprising stimulating them with an adhesin. A cytokine may also be provided to prime the dendritic cells. In vivo the cytokine may already be present, thus exogenous cytokine may not be required. However, if the DCs are being stimulated in vitro, it may be necessary to provide a cytokine to prime the DCs. The cytokine and adhesin may be administered simultaneously or sequentially, and when administered sequentially, administration may occur in either order. The invention also provides a composition comprising a cytokine and an adhesin and the use of such a composition as an immunopotentiator.

The invention also provides a composition comprising an adhesin, an antigen and/or immunogenic composition, and optionally one or more additional adjuvants and/or immunopotentiators. Additional adjuvants and/or immunopotentiators are known in the art, and include, but are not limited to, immunostimulatory oligonucleotides, such as CpG; MF59 and other oil-in-water emulsions; alum and other mineral salts; ISCOMS; imidazoquinoline compounds such as R-848; and the like. Additional general categories of adjuvants that can be used in the compositions of the invention include mineral salts, bacterial or microbial derivatives such as e.g., LPS and Lipid A derivatives, saponin compositions, bioadhesives and mucoadhesives, microparticles, liposomes, polyoxyethylene ether and polyoxyethylene ester formulations, PCPP, muramyl peptides and imidazoquinoline compounds.

The invention also provides adhesins for use as immunopotentiators, e.g., for use in adjuvanting vaccinations.

### Adhesins

Adhesins are virulence associated antigens on pathogens that are involved in adhesion. The adhesins used in some embodiments of the invention bind a receptor on the surface of dendritic cells. Preferably the adhesin can bind to heparin. Preferably the adhesin has the ability to bind to glycosaminoglycans such as heparin, e.g., the adhesin may comprises a heparin-binding domain. Such knowledge allows screening assays to be set up to search for new adhesins, or other binding analogues, potentially useful as adjuvants in stimulating innate immunity.

One example of an adhesin is NadA. NadA (NMB1994; Q9JXK7; GI:81784145, SEQ ID NO: 1) was first isolated from the meningococcus B strain MC58 [3]. Four different forms of NadA have been described which are obtained from allele 1 (362 amino acids, SEQ ID NO: 2), allele 2 (398 amino acids, SEQ ID NO: 3), allele 3 (405 amino acids, SEQ ID NO: 4) or allele 4 (323 amino acids, AAS75121.1, GI:45649061, SEQ ID NO: 5). It is postulated that in addition to the adhesion role, NadA may interfere with the activation of the alternative pathway of the complement system, specifically in humans, as well as interfering with opsonization. Without being limited to a particular hypothesis, the interference with complement activation may be due to NadA's binding to heparin.

Adhesins are well known in the art. For example, reference 4 describes a number of adhesins which are homologues of NadA from species including H. aegyptius, A. actinomycetemcomitans and H. somnus. Other homologues of NadA include the YadA protein of Yersinia entercolotica [5] and the UspA2 protein of Moraxella catarrhalis [6].

Other adhesins known in the art include the Mycoplasma pirum P1-like adhesin [7], the Entamoeba histolytica GalNAc-inhibitable adhesin [8], various Escherichia coli expressed virulence factors [9] such as the K88 fibrillae protein [10] and the 987P fimbriae protein [11], the Anaplasma marginale MSP1a and 1b polypeptides [12], the Trichomonas foetus adhesin [13], the group A Streptococcus protein M and MSCRAMM™s [14-18].

Fragments of these adhesins may also be used in the composition or method of the invention. Fragments include the various domains of adhesin proteins, such as the globular head, the coiled coil region and the transmembrane anchor region.

Preferred fragments retain DC binding activity.

Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25, 45 or more) from the N-terminus of the adhesin amino acid sequence. In particular, preferred fragments omit at least the N-terminus leader sequence (and the omitted leader sequence may be replaced by a heterologous leader sequence).

Other preferred fragments omit one or more (i.e. 1, 2, or 3) of structural domains of the adhesin. Other preferred fragments consist of one or more (i.e. 1, 2, or 3) of the structural domains of the adhesin. Preferred fragments lack the membrane anchor. Preferably the fragments are soluble.

Preferred adhesin polypeptides are presented in oligomeric form (e.g. dimers, trimers, tetramers, etc.). Trimers are preferred, but monomeric polypeptides of the invention are also useful.

A particularly preferred fragment of NadA is NadAΔ351-405 (SEQ ID NO: 18; also known as 961cL), which is a soluble secreted recombinant mutant which lacks the membrane anchor. The WT NadA protein usually forms oligomers anchored to the surface of the bacteria whereas 961cL does not.

The polypeptides may be prepared by various means e.g. by chemical synthesis (at least in part), by digesting longer polypeptides using proteases, by translation from RNA, by purification from cell culture, (e.g. from recombinant expression or from, for example, N. meningitidis culture) etc. Polypeptides are preferably prepared in a substantially pure or substantially isolated form (i.e. substantially free from other Neisserial or host cell proteins). In general, the polypeptides are provided in a non-naturally occurring environment e.g. they are separated from their naturally occurring environment. In certain embodiments the polypeptide is present in a composition that is enriched for the polypeptide as compared to a control. As such, purified polypeptide is provided, whereby purified is meant that the polypeptide is present in a composition that is substantially free of other expressed polypeptides, whereby substantially free is meant that less than 50%, usually less than 30% and more usually less than 10% of the composition is made up of other expressed polypeptides.

### Cytokines

Various cytokines may be used in the methods and compositions of the invention. For example, interleukins such as IL-21, IL-12, IL-18, IL-15 and interferons may be used. Preferably the cytokine used in the invention is an interferon (IFN). More preferably, the cytokine is IFN-γ.

### Dendritic Cells

Dendritic cells are antigen presenting cells which have the ability to prime naive T lymphocytes to antigens. All naive T cells require two signals for activation to elicit an immune response. For CD8⁺ lymphocytes (CTLs), the first signal, which imparts specificity, consists of presentation to the CD8⁺ cell of an immunogenic peptide fragment (epitope) of the antigen bound to the Class I MHC (HLA) complex present on the surface of antigen-presenting cells (APCs) such as dendritic cells. This complex is recognized specifically by a T cell antigen receptor (TCR), which communicates the signal intracellularly.

Binding to the T cell receptor is necessary but not sufficient to induce T cell activation, and usually will not lead to cell proliferation or cytokine secretion. Complete activation requires a second co-stimulatory signal(s). These signals serve to further enhance the activation cascade. Among the co-stimulatory molecules on antigen-presenting cells, B7 and cell adhesion molecules (integrins) such as ICAM-1 assist in this process by binding to CD28 and LFA-1, respectively, on the T cell. When a CD8⁺ cell interacts with an antigen-presenting cell bearing an immunogenic peptide (epitope) bound by a Class I MHC molecule in the presence of appropriate co-stimulatory molecule interactions, the CD8⁺ cell becomes a fully activated cytolytic T cell.

Dendritic cells (DCs) for use in the invention may be Langerhans cells (LCs), tissue DCs, blood DCs, interdigitating DCs, thymic DCs, or follicular DCs. Preferably the DCs are blood DCs. Particularly preferred DCs are myeloid blood CD11c⁺DCs and monocyte-derived DCs (Mo-DCs) which are derived from CD16⁺CD14⁺ or CD2⁺CD14⁺ precursor monocytes.

### Sensitisation of Dendritic Cells

Following (or during) activation of dendritic cells by the methods of some embodiments of the invention, the dendritic cells may be incubated with one or more antigens that are characteristic of one or more diseases or pathogens. For example, the use of prostate specific membrane antigen and peptides thereof (PSM-P1 and PSM-P2) for sensitising dendritic cells has been described [19].

Such loaded DCs may then be administered to a host where the specific antigen is presented by the loaded DCs to the immune system. Thus, by loading DCs with specific antigens, is it possible to raise specific immune responses directed towards a given antigen or epitope on a pathogen or disease (such as cancer). This activates the immune system against that particular antigen, epitope or disease.

Preferably the antigen or epitope is obtained from a cancer tumour [20], preferably, renal cell carcinoma [21], multiple myeloma [22], lymphoma [23], malignant melanoma or other melanomas [24, 25] such as metastatic melanomas, melanomas derived from either melanocytes or melanocytes related nevus cells, melanosarcomas, melanocarcinomas, melanoepitheliomas, melanoma in situ superficial spreading melanoma, nodular melanoma, lentigo maligna melanoma, acral lentiginous melanoma, invasive melanoma or familial atypical mole and melanoma (FAM-M) syndrome. Such melanomas in mammals may be caused by, chromosomal abnormalities, degenerative growth and developmental disorders, mitogenic agents, ultraviolet radiation (UV), viral infections, inappropriate tissue expression of a gene, alterations in expression of a gene, and presentation on a cell, or carcinogenic agents. Preferably the cancer being treated is breast, stomach, ovarian, colon, salivary gland, liver, kidney, lung, head and neck, nasopharyngeal, bladder, cervical, gastric or prostate cancer [26]. Examples of peptides from breast and ovarian cancers that may be used for sensitising DCs are given in ref 27. The antigen or epitope may be derived from a HER-2 polypeptide (as described in ref. 28).

External antigens derived from pathogens may also be used to sensitise the DCs. Such antigens may be derived from pathogens such as viral agents including, but not limited to, human immunodeficiency virus (HIV), hepatitis B virus (HBV), influenza, human papilloma virus (HPV), foot and mouth (coxsackieviruses), the rabies virus, herpes simplex virus (HSV), and the causative agents of gastroenteritis, including rotaviruses, adenoviruses, caliciviruses, astroviruses and Norwalk virus; bacterial agents including, but not limited to, E. coli, Salmonella thyphimurium, Pseudomonas aeruginosa, Vibrio cholerae, Neisseria gonorrhoeae, Helicobacter pylori, Hemophilus influenzae, Shigella dysenteriae, Staphylococcus aureus, Mycobacterium tuberculosis and Streptococcus pneumoniae, fungal agents and parasites such as Giardia.

Alternatively, RNA encoding or a plasmid vector encoding such an antigen can be transfected into the DC. Similarly, nonreplicating recombinant viral vectors expressing such an antigen can be transduced into the DC.

Immunogenicity may be further enhanced by using antigens coupled to or expressing other immunogenic proteins such as keyhole limpet hemocyanin, cytokines (IL-12, IL-15), costimulatory molecules (B7-2, CD40L) or chemokines (e.g. CCL21).

### Knock-Out Dendritic Cells

Alternatively it is possible to stimulate DCs that are unable to provide the second signal required by T cells for activation (through the interaction of CD28/CD86). This results in tolerisation of the T cells, resulting in anergy [see ref. 29]. Thus the invention provides a method of activating a CD86⁻ DC, comprising stimulating the DC with an adhesin.

Such activated DCs that are unable to provide the second signal required for T cell activation can be loaded with autoimmune antigens. Thus, anergy is induced in the T cell population that recognises that autoimmune antigen, resulting in a decrease or cessation in the autoimmune response. Autoimmune antigens that may be used to sensitise the DCs include those derived from multiple sclerosis, Alzheimer's disease, rheumatoid arthritis, coeliac disease, diabetes mellitus. Similarly antigens may be derived from graft tissue, thus helping to prevent host-graft rejection.

### Immunopotentiation Compositions

Some embodiments of compositions according to the invention comprise an adhesin. In some embodiments, the composition may further comprise a cytokine. In some embodiments, the composition may further comprise a sensitising antigen, for example, an exogenous antigen. Preferably, the cytokine is an interferon, preferably IFN-γ. Preferably the adhesin is NadA. Compositions may also comprise a co-stimulatory compound such as:
An imidazoquinoline compound, such as Imiquimod (“R-837”) [30,31], Resiquimod (“R-848”) [32], and their analogs; and salts thereof (e.g. the hydrochloride salts). Further details about immunostimulatory imidazoquinolines can be found in references 33 to 37. Preferably, R-848 is used.
An immunostimulatory oligonucleotide, such as one containing a CpG motif (a dinucleotide sequence containing an unmethylated cytosine linked by a phosphate bond to a guanosine), or a double-stranded RNA, or an oligonucleotide containing a palindromic sequence, or an oligonucleotide containing a poly(dG) sequence.
Immunostimulatory oligonucleotides can include nucleotide modifications/analogs such as phosphorothioate modifications and can be double-stranded or (except for RNA) single-stranded. References 38, 39 and 40 disclose possible analog substitutions e.g. replacement of guanosine with 2′-deoxy-7-deazaguanosine. The adjuvant effect of CpG oligonucleotides is further discussed in refs. 41-46. A CpG sequence may be directed to TLR9, such as the motif GTCGTT or TTCGTT [47]. The CpG sequence may be specific for inducing a Th1 immune response, such as a CpG-A ODN (oligodeoxynucleotide), or it may be more specific for inducing a B cell response, such a CpG-B ODN. CpG-A and CpG-B ODNs are discussed in refs. 48-50. Preferably, the CpG is a CpG-A ODN. Preferably, the CpG oligonucleotide is constructed so that the 5′ end is accessible for receptor recognition. Optionally, two CpG oligonucleotide sequences may be attached at their 3′ ends to form “immunomers”. See, for example, references 47 & 51-53. A useful CpG adjuvant is CpG7909, also known as ProMune™ (Coley Pharmaceutical Group, Inc.).
As an alternative, or in addition, to using CpG sequences, TpG sequences can be used [54]. These oligonucleotides may be free from unmethylated CpG motifs.
The immunostimulatory oligonucleotide may be pyrimidine-rich. For example, it may comprise more than one consecutive thymidine nucleotide (e.g. TTTT, as disclosed in ref. 54), and/or it may have a nucleotide composition with >25% thymidine (e.g. >35%, >40%, >50%, >60%, >80%, etc.). For example, it may comprise more than one consecutive cytosine nucleotide (e.g. CCCC, as disclosed in ref. 54), and/or it may have a nucleotide composition with >25% cytosine (e.g. >35%, >40%, >50%, >60%, >80%, etc.). These oligonucleotides may be free from unmethylated CpG motifs.
Immunostimulatory oligonucleotides will typically comprise at least 20 nucleotides. They may comprise fewer than 100 nucleotides.
LPS or a derivative thereof, in particular monophosphoryl lipid A or a derivative thereof, in particular 3-O-deacylated monophosphoryl lipid A (‘3dMPL’, also known as ‘MPL™’) [55-58]. 3dMPL (also known as 3 de-O-acylated monophosphoryl lipid A or 3-O-desacyl-4′-monophosphoryl lipid A) is an adjuvant in which position 3 of the reducing end glucosamine in monophosphoryl lipid A has been de-acylated. 3dMPL has been prepared from a heptoseless mutant of Salmonella minnesota, and is chemically similar to lipid A but lacks an acid-labile phosphoryl group and a base-labile acyl group. It activates cells of the monocyte/macrophage lineage and stimulates release of several cytokines, including IL-1, IL-12, TNF-α and GM-CSF (see also ref. 59). Preparation of 3dMPL was originally described in reference 60.
3dMPL can take the form of a mixture of related molecules, varying by their acylation (e.g. having 3, 4, 5 or 6 acyl chains, which may be of different lengths). The two glucosamine (also known as 2-deoxy-2-amino-glucose) monosaccharides are N-acylated at their 2-position carbons (i.e. at positions 2 and 2′), and there is also O-acylation at the 3′ position. The group attached to carbon 2 has formula —NH—CO—CH₂—CR¹R^{1′}. The group attached to carbon 2′ has formula —NH—CO—CH₂—CR²R^{2′}. The group attached to carbon 3′ has formula —O—CO—CH₂—CR³R^{3′}. A representative structure is:

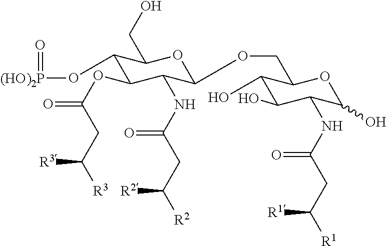

Groups R¹, R² and R³ are each independently —(CH₂)ₙ—CH₃. The value of n is preferably between 8 and 16, more preferably between 9 and 12, and is most preferably 10.
Groups R^{1′}, R^{2′} and R^{3′} can each independently be: (a) —H; (b) —OH; or (c) —O—CO—R⁴, where R⁴ is either —H or —(CH₂)ₘ—CH₃, wherein the value of in is preferably between 8 and 16, and is more preferably 10, 12 or 14. At the 2 position, m is preferably 14. At the 2′ position, m is preferably 10. At the 3′ position, m is preferably 12. Groups R^{1′}, R^{2′} and R^{3′} are thus preferably —O-acyl groups from dodecanoic acid, tetradecanoic acid or hexadecanoic acid.
When all of R^{1′}, R^{2′} and R^{3′} are —H then the 3dMPL has only 3 acyl chains (one on each of positions 2, 2′ and 3′). When only two of R^{1′}, R^{2′} and R^{3′} are —H then the 3dMPL can have 4 acyl chains. When only one of R^{1′}, R^{2′} and R^{3′} is —H then the 3dMPL can have 5 acyl chains. When none of R^{1′}, R^{2′} and R^{3′} is —H then the 3dMPL can have 6 acyl chains. The 3dMPL adjuvant used according to the invention can be a mixture of these forms, with from 3 to 6 acyl chains, but it is preferred to include 3dMPL with 6 acyl chains in the mixture, and in particular to ensure that the hexaacyl chain form makes up at least 10% by weight of the total 3dMPL e.g. ≧20%, ≧30%, ≧40%, ≧50% or more. 3dMPL with 6 acyl chains has been found to be the most adjuvant-active form.
Thus the most preferred form of 3dMPL for inclusion in compositions of the invention is:

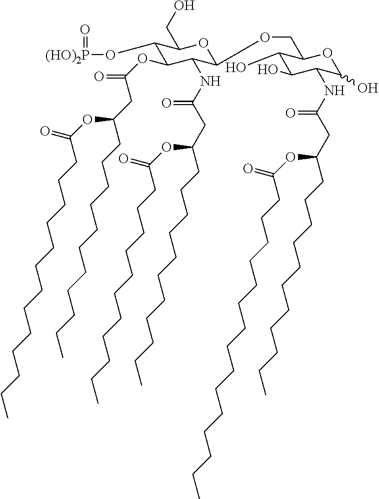

Where 3dMPL is used in the form of a mixture then references to amounts or concentrations of 3dMPL in compositions of the invention refer to the combined 3dMPL species in the mixture.
In aqueous conditions, 3dMPL can form micellar aggregates or particles with different sizes e.g. with a diameter <150 nm or >500 nm. Either or both of these can be used with the invention, and the better particles can be selected by routine assay. Smaller particles (e.g. small enough to give a clear aqueous suspension of 3dMPL) are preferred for use according to the invention because of their superior activity [61]. Preferred particles have a mean diameter less than 220 nm, more preferably less than 200 nm or less than 150 nm or less than 120 nm, and can even have a mean diameter less than 100 nm. In most cases, however, the mean diameter will not be lower than 50 nm. These particles are small enough to be suitable for filter sterilization. Particle diameter can be assessed by the routine technique of dynamic light scattering, which reveals a mean particle diameter. Where a particle is said to have a diameter of x nm, there will generally be a distribution of particles about this mean, but at least 50% by number (e.g. ≧60%, ≧70%, ≧80%, ≧90%, or more) of the particles will have a diameter within the range x±25%.
3dMPL can advantageously be used in combination with an oil-in-water emulsion. Substantially all of the 3dMPL may be located in the aqueous phase of the emulsion.
The 3dMPL can be used on its own, or in combination with one or more further compounds. For example, it is known to use 3dMPL in combination with the QS21 saponin [62] (including in an oil-in-water emulsion [63]), with an immunostimulatory oligonucleotide, with both QS21 and an immunostimulatory oligonucleotide, with aluminum phosphate [64], with aluminum hydroxide [65], or with both aluminum phosphate and aluminum hydroxide.

Further, in some embodiments, compositions of the invention comprise dendritic cells that have been stimulated with a cytokine and an adhesin and then sensitised by incubation with a disease antigen. The components may be present as polypeptides and/or as nucleic acid molecules encoding polypeptides with the appropriate expression signals, as will be recognized by one of skill in the art.

Compositions of the invention may further comprise DC mobilization factors, tumor cell apoptotic agent and/or necrotic agents (tumor killing agents), DC maturation agents, T cell enhancing agents and chemoattractants.

Examples of such mobilisation factors are GM-CSF, mutants and fusion proteins thereof [66,67] and IL-15. Examples of tumour killing agents include various members of the Tumor Necrosis Factor (TNF) superfamily (including TNF, Lymphotoxins alpha and beta, CD40L, and TNF-related apoptosis-inducing or TRAIL), chemotherapeutic agents and radiotherapeutic agents.

Chemoattractants that may be used include the chemokines MCPs 1-5, MIP-1 alpha or beta, RANTES or eotaxin as well as MIP-3 alpha, MIP-3 beta, MIP-5, MDC, SDF-1, and the cytokines IL-1, TNF-alpha and IL-10.

The compositions may further comprise anti-tumour antibodies such as rituximab, trastuzumab [68], IMC-C225 [69] and ABX-EGF [70].

Some tumor secretions can interfere with the function of the mature DC. For example, some tumors (e.g., melanoma) secrete a cytokine (IL-10) that prevents generation and accumulation of DCs and antitumor activity by the DCs. Thus compositions of the invention may include an IL-10 inhibitor.

The compositions of the invention may comprise other active agents, such as one or more anti-inflammatory agent(s), anti-coagulant(s) and/or human serum albumin (preferably recombinant).

The compositions may be suitable for administration by injection (e.g. into the blood). Intravenous injection is preferred, but local or topical routes of administration may also be used in some embodiments. For intravenous injection, the hepatic portal vein is a preferred route. Thus, in some embodiments, the invention provides a syringe containing a composition(s) of the invention.

The composition may be essentially in the form in which the cells and/or other components exit culture. However, the cells and/or other components may be treated between culture and administration. For instance, the cells may be irradiated prior to administration e.g. to ensure that the cells cannot divide.

The composition may comprise a pharmaceutical carrier. This carrier may comprise a cell culture medium which supports the cells' viability. The medium will generally be serum-free in order to avoid provoking an immune response in a recipient. The medium is preferably free from animal-derived products (e.g. BSA). The carrier may be buffered and/or pyrogen-free. Compositions may be presented in vials, or they may be presented in ready-filled syringes. The syringes may be supplied with or without needles. A syringe may include a single dose of the composition, whereas a vial may include a single dose or multiple doses. Injectable compositions will usually be liquid solutions or suspensions. Alternatively, they may be presented in solid or lyophilized form (e.g. cryogenically frozen for thawing prior to injection).

Compositions of the invention may be packaged in unit dose form or in multiple dose form. For multiple dose forms, vials are preferred to pre-filled syringes. Effective dosage volumes can be routinely established, but a typical human dose of the composition for injection has a volume of 0.5 ml. The dose may be 0.1 to 10 ml, preferably 0.25 to 8 ml, preferably 0.5 to 5 ml, preferably 0.75 to 3 ml, preferably 1 to 2 ml.

The invention also provides a composition of the invention for use as a medicament. The medicament is preferably able to raise an immune response in a mammal (i.e. it is an immunogenic composition).

Compositions of the invention may be administered as part of a treatment regime that includes one or more of chemotherapy, radiotherapy, surgery (including cryo-surgery), photodynamic therapy, gene therapy and hyperthermia.

In some embodiments, the invention provides a composition according to the invention for use in therapy.

In some embodiments, the invention also provides the use of a composition of the invention (and other optional antigens) in the manufacture of a medicament for raising an immune response in a mammal. The medicament is preferably a vaccine.

In some embodiments, the invention also provides a method for raising an immune response in a mammal comprising the step of administering an effective amount of a composition of the invention. The immune response is preferably protective and preferably involves antibodies. The method may raise a booster response.

The mammal is preferably a human. Where the vaccine is for prophylactic use, the human is preferably a child (e.g. a toddler or infant); where the vaccine is for therapeutic use, the human is preferably an adult or an adolescent. A vaccine intended for children may also be administered to adults e.g. to assess safety, dosage, immunogenicity, etc.

In some embodiments, the subject being treated is refractive to other forms of therapy. For example, if the composition is for use in treating cancer, the patient may have undergone surgery or radiotherapy to remove a tumor.

If used for treating cancer, a composition according to the invention may be administered before, after or concurrently with another form of therapy such as radiotherapy, chemotherapy, photodynamic therapy or surgery (including cryo-surgery).

In some embodiments, the invention also provides a method of making a vaccine comprising activating dendritic cells with an adhesin and then loading the DCs with a disease or pathogen derived peptide

In some embodiments, the invention provides activated DCs suitable for administration to a subject wherein DCs, which were isolated from that subject have been stimulated with an adhesin.

In some embodiments, the invention provides a method of raising an immune response in a subject comprising obtaining immature dendritic cells from a subject, activating the DCs with an adhesin, (optionally) loading the activated dendritic cells with a disease or pathogen derived peptide and returning the activated DCs to the subject.

If the composition is administered to reduce an anti-grail response, the composition may be administered before the graft (i.e. pre-tolerisation) or at substantially the same time. It is preferred to administer the cells before the graft (e.g. at least 1 day before, preferably at least 3 days before, and typically at least 5, 6, 7, 8, 9 or 10 days before).

In some embodiments, the invention provides screening methods for searching for candidate immunopotentiators. For example, substances that bind low and/or high affinity NadA binding sites of dentritic cells may be obtained using methods known to those of skill in the art, based on the teachings provided herein. Such substances may be adhesins, other pathogenic proteins, protein fragments, or small molecule binding analogs that may be obtained, e.g., from natural or synthetic sources, including, e.g., from combinatorial libraries.

### BRIEF DESCRIPTION OF DRAWINGS

FIG. 1A shows the effect of Neisseria meningitidis NadAΔ351-405 on dendritic cell morphology. Monocyte-derived DCs were cultured for 18 h at 37° C. with INF-γ (1000 U/ml) or with no priming agent, and then further stimulated for 3 h with NadA 1.5 μM or E. coli LPS 1 μg/ml as indicated. Light microscopy images are representative of one of several experiments. FIG. 1B shows the same effect for human macrophages. FIG. 1C shows the effect of stimulation with E. coli OMV on (A) macrophages and (B) monocytes. FIG. 1D shows the effect of stimulation with N. meningitidis OMV on (A) macrophages and (B) monocytes.

FIG. 2 shows the expression of maturation markers on NadAΔ351-405 stimulated mo-DCs, subjected or not to INF-γ priming. Data correspond to the expression, determined by indirect labelling with anti-CD antibodies and flow cytofluorometry, of indicated specific cell surface molecules on mo-DCs pre-treated for 18 h with INF-γ 1000 U/ml (filled bars) or not (open bars) and pulsed for 24 h with NadAΔ351-405 1.5 μM, with E. coli LPS 1 μg/ml or with no agonist (ctrl), as indicated. Values are the mean fluorescence intensity (MFI) ±SD obtained from five independent experiments run in duplicate.

FIG. 3 shows the effect of Neisseria meningitidis NadAΔ351-405 on cytokine and chemokine secretion by mo-DCs, subjected or not to INF-γ priming. Cells were treated (filled bars) or not (open bars) for 18 h with INF-γ (1000 U/ml) and further incubated with no agonists (ctrl), NadA 1.5 μM or E. coli LPS 1 μg/ml. ELISA (IL-12p40) and Bioplex multiplex cytokine assay (IL-6, TNFα, IL-8, IL-10, IL12-p70) were performed on culture supernatants collected after 24 h. Data are mean antigen concentrations in the supernatants (pg/ml/0.5×10⁶ cells) ±SD from five donors. Numbers on top of bars are the percent of cytokine production, compared to maximal production due to LPS stimulation after INF-γ priming.

FIG. 4 shows the kinetics of IL-6, TNFα, IL23-p19, IL12-p35, IL12/IL23-p40 mRNA expression levels. Mo-DCs were primed (+) or not (−) with INF-γ before NadA (1.5 μM) or LPS (1 μg/ml) stimulation. The amount of mRNA encoding the indicated cytokines was analysed by quantitative cybr-green RT-PCR at hours 3, 5 and 8. Control corresponded to untreated cells. Absolute concentrations of cytokine cDNA copies were calculated by comparison with appropriate standards, and normalised to the housekeeping gene HMBS. One representative experiment of three is shown.

FIG. 5 shows the binding of Alexa-NadAΔ351-405 to mo-DCs. A) Cells pre-treated for 18 h with INF-γ 1000 U/ml (solid symbols) or with medium alone (open symbols) were incubated for 3 h with increasing concentration of Alexa-NadAΔ351-405 at 37° C. (square symbols) or at 0° C. (triangular symbols). B) Scatchard plot analysis of binding data reported in panel A. Kd₁ and Kd₂ indicate high and low affinity binding sites, respectively. Data are from an experiment representative of four. C) Representative flow cytometric profiles of mo-DCs stimulated for 3 h with the indicated concentrations of Alexa-NadA (thin line) or pre-treated for 18 h with INF-γ 1000 U/ml and further pulsed for 3 h with the same Alexa-NadA concentrations (thick line). Grey histograms represent MFI of control cells.

FIG. 6 shows the dose-response analysis of NadAΔ351-405 on mo-DCs. A) Data compare CD86 plasma membrane expression and indicated cytokine and chemokine secretion by mo-DCs stimulated with different concentrations (37.5-3800 nM) of NadA for 24 hours (data represented as scattered symbols), and Alexa-NadAΔ351-405 binding curve (data SD, interpolated by a black line). Solid symbols corresponded to mo-DCs primed for 18 h with INF-γ (1000 U/ml) while open symbols to non-primed cells. NadA concentrations are represented in a logarithmic scale to better show in the same graph the effects of high affinity (Kd₁=90 nM) and low affinity (Kd₂=4 μM) NadA-cell interactions. B) Dose-dependence of the distribution profile of CD86 surface expression by mo-DCs stimulated with the indicated concentrations of NadA. Data from mo-DCs primed with INF-γ before NadA are shown by thin lines, whereas cells treated only with NadA are indicated by thick lines. Gray-filled histograms represent cell surface CD86 expression on untreated cells and gray lines show CD86 expression following 18 h stimulation with INF-γ alone. Results are from one donor and are representative of similar data obtained from experiments carried out with mo-DCs from three different donors.

FIG. 7 shows the activation of allogenic naive Th lymphocytes by INFγ-primed mo-DCs matured with NadAΔ351-405. A) Increasing numbers of mo-DCs primed or not with INF-γ, as indicated, and treated with medium alone (open round symbols), with NadA 1.5 μM (square symbols) or LPS 1 μg/ml (solid round symbols) were co-cultured with purified naive CD45RA⁺CD4⁺ T cells (0.03×10⁶ cells/well). After 5 days T-cell proliferation was assessed by [³H] thymidine incorporation for 6 h. Results are the mean±SD of triplicate values, from three independent experiments. B) Cytokine-driven differentiation of naïve T cells. CD4+ naïve T cells were co-cultured at 1:30 stimulator/responder ratio with allogenic irradiated DCs stimulated as previously described. After 6 h with PMA (10 ng/ml) and ionomycin (1 μg/ml) 10⁴ cells were analysed by flow cytometry for INF-γ and IL-4 intracellular expression. The percentage of positive cells is indicated in the quadrants. Data are from one representative experiment of two performed. C) Cytokine profile of T effectors co-cultured at 1:300-1:100-1:30 ratios. Human naïve CD4⁺ T cells after 5 days co-culture with allogenic irradiated DCs INF-γ primed before NadA (1.5 μM) or LPS (1 μg/ml) stimulation were restimulated with PMA and ionomycin for 5 h. The figures show the percentage of INF-γ, IL-4 and INF-γ/IL-4 producing cells, as indicated. Data are representative of two independent experiments, performed with cells from different donors.

FIG. 8 shows specific binding of NadAΔ351-405 to mo-DCs. Chang and CHO-K1 cells were incubated for 3 h at 37° C. with Alexa⁴⁸⁸-labeled NadAΔ351-405 (250 nM) in the presence or the absence of non-labelled NadAΔ351-405 (0, 1, 2.5 or 5 μM), washed and analyzed by FACS; results shown are the relative MFI values ±SE, n=3.

FIG. 9 shows (A) CD86 expression and (B) IL12p70 production after stimulation with NadAΔ351-405 or common PAMP stimuli. Mo-DCs were treated (open bars) or not (filled bars) for 18 h with IFN-γ (1000 U/ml) and further incubated with different indicated concentration of NadAΔ351-405, flagellin, CpG2216 oligodeoxynucleotide or LPS. CD86 expression was determined after 24 h incubation by labelling with anti-CD86 antibody and flow cytometry analysis. Mean fluorescence intensity (MFI) ±SE obtained from five independent experiments are shown. ELISA (IL-12p70) assay was performed on culture supernatants collected after 24 h. Data are mean antigen concentration in the supernatants ±SE from six donors. Significance of values (P≦0.05) compared to control samples, is indicated by an asterisk.

FIG. 10 shows R-848 co-stimulation enhances IL-12p70 secretion by NadA-treated mo-DCs. Mo-DCs treated or not for 18 h with IFN-γ (1000 U/ml) were incubated for further 24 h with NadAΔ351-405 (1.5 μM), flagellin (10 μg/ml), CpG non-methylated DNA (10 μg/ml) or LPS (0.1 and 100 ng/ml) in the absence (shaded bars) or presence (open bars) of R-848 (1 μM). CD86 was determined by flow cytometry analysis and 11,12p70 in the supernatants was quantified by ELISA. Results are expressed as mean±SE of six experiments. Significance of values (P≦0.05) compared to control samples, is indicated by an asterisk.

FIG. 11 shows the analysis of NadA_{Δ351-405} binding to leukocyte populations. Samples of human blood, after hemolysis were incubated with NadA_{Δ351-405}-Alexs 600 nM for 3 hours at 37° C., then incubated with phycoerythrin-conjugated monoclonal antibodies specific for the different cell populations (PE). The analysis was performed through flow cytometry, excluding dead cells and cell debris positive to the propidium iodine. (A) In the Dot-plots, values are reported for the percentage of cells present in the selected quadrant. (B) The histogram shows the measured mean fluorescent intensities (MFI) for the different samples.

FIG. 12 shows the analysis of NadA binding to monocytes. The graphs plot the mean fluorescence intensities (MFI) ±SD measured in monocytes that have been incubated with NadA_{Δ351-405}-Alexa, at different concentrations (A), or with 100 nM NadA_{Δ351-405}-Alexa in presence of increasing concentrations of unlabelled protein (B), for 3 hours at 37° C. or 0° C. The reported data are the average of three independent experiments repeated in triplicate.

FIG. 13 shows the analysis of NadA binding to human macrophages. The graphs plot the mean fluorescence intensities (MFI) ±SD measured in human macrophages that have been incubated with NadA_{Δ351-405}-Alexa, at different concentrations (A), or with 100 nM NadA_{Δ351-405}-Alexa in presence of increasing concentrations of unlabelled protein (B), for 3 hours at 37° C. or 0° C. The reported data are the average of three independent experiments repeated in triplicate.

FIG. 14 shows a western blot analysis of E. coli OMV. (A) Western blot for total bacterial lysate, (B) Western blot for NadA.

FIG. 15 shows the analysis of human monocyte surface markers CD80, CD86 and HLA-DR.

FIG. 16 shows the analysis of human macrophage surface markers CD80, CD86, HLA-DR and ICAM-1.

FIGS. 17 and 19 show the analysis of human monocyte surface markers CD80, CD86, HLA-DR and ICAM-1 in the presence of OMV from E. coli or N. meningitidis, respectively.

FIGS. 18 and 20 show the analysis of human macrophage surface markers CD80, CD86, HLA-DR and ICAM-1 in the presence of OMV from E. coli or N. meningitidis, respectively.

FIGS. 21 and 22 show the analysis of IL-1α, IL-1β and TNFα secretion in human monocytes and macrophages, respectively.

FIGS. 23 and 24 show the analysis of IL-6 and GM-CSF secretion in human monocytes and macrophages, respectively.

FIGS. 25 and 26 show the analysis of IL-12(p40), IL-12(p70) and IL-23 secretion in human monocytes and macrophages, respectively.

FIG. 27 shows the analysis of IL-10 secretion in human monocytes and macrophages.

FIGS. 28 and 29 show the analysis of IL-8, MCP-1, RANTES, EOTAXIN and MIP-1α secretion in human monocytes and macrophages, respectively.

FIG. 30 shows the analysis of IL-1α, IL-1β and TNFα secretion in human monocytes and macrophages.

FIG. 31 shows the analysis of IL-6 and GM-CSF secretion in human monocytes and macrophages.

FIGS. 32 and 33 show the analysis of IL-10. IL-12(p40), IL-12(p70) and IL-23 secretion in human monocytes and macrophages, respectively.

FIG. 34 shows the analysis of IL-8, MCP-1, IP-10 and RANTES secretion in human monocytes.

FIG. 35 shows the analysis of IL-8, MCP-1 and IP-10 secretion in human macrophages.

FIG. 36 shows the analysis of IL-1α, IL-1β and TNFα secretion in human monocytes and macrophages.

FIG. 37 shows the analysis of IL-6 secretion in human monocytes and macrophages.

FIGS. 38 and 39 show the analysis of IL-10, IL-12(p40), IL-12(p70) and IL-23 secretion in human monocytes and macrophages, respectively.

FIG. 40 shows the analysis of IL-8, IL-10, RANTES and MCP-1 secretion in human monocytes.

FIG. 41 shows the analysis of IL-8, IL-10, MIP-1α and MCP-1 secretion in human macrophages.

FIG. 42 shows the apoptosis and survival analysis of NadA-treated monocytes. A) Caspase-3 assay, B) MTT assay.

FIG. 43 shows the morphological analysis of NadA treated monocytes.

FIG. 44 shows the analysis of human monocyte surface markers CD80, CD86, HLA-DR and ICAM-1.

FIG. 45 shows the analysis of cytokine and chemokine secretion in human monocytes.

### MODES FOR CARRYING OUT THE INVENTION

### NadA

Soluble recombinant NadA was designed and purified as previously described [71]. Briefly, the DNA sequence of NadA allele 3, cloned from the hypervirulent N. meningitidis B strain 2996, encoding the deletion mutant NadAΔ351-405, with no membrane anchor, was cloned into a pET21b vector (Novagen). The protein secreted in the extracellular medium of the transformed E. coli BL21(DE3)-NadAΔ351-405 strain was purified by Q Sepharose XL and Phenyl Sepharose 6 Fast Flow (Pharmacia) chromatography. LPS contamination (tested by Limulus test kit from Sigma) was ablated to less than 0.005 EU/mg of protein by a further passage on Hydroxyl apatite ceramic column (HA Macro. Prep). No E. coli antigens were detected by western immunoblot analysis with a rabbit polyclonal antibody raised against whole E. coli cells (Dako). Purified NadAΔ351-405 shows a single 35 KDa band after SDS-PAGE and silver staining, consistent with the predicted molecular weight, and is a homo-trimer, as assessed by light scattering analysis. Aliquots of protein solution (2 mg/ml in PBS, pH 7.4) were frozen in liquid nitrogen and stored at −80° C.

### Labelling of NadAΔ351-405

NadA was conjugated to the fluorescent probe Alexa 488 using a N-hydroxysuccinimidyl derivative (Molecular Probes Inc.) according to the manufacturer's instructions. Alexa-NadAΔ351-405 was separated from left reagents by size exclusion chromatography using Sephadex G25 (Sigma) columns pre-equilibrated and eluted with PBS at room temperature.

### Cell Isolation and Culture Conditions

Reagents used were tested for low endotoxin contamination using the Limulus amoebocyte assay (Sigma). Dendritic cells were generated from human peripheral blood mononuclear cells (PBMC) as described previously [72]. In brief, PBMC were isolated from buffy coats of healthy donors by Ficoll-Paque Plus density gradient centrifugation (Amersham Pharmacia Biotech AB). Separate monocyte and T-cell fractions were obtained from PBMCs by Percoll density gradient centrifugation (Amersham Pharmacia Biotech AB). Residual T and B cells were removed from monocyte fraction by plastic adherence of 3×10⁶ cells per well in 6-well plates (Costar) resulting in CD14⁺ monocyte populations of >95% purity (determined by flow cytometry). DC were obtained by 6-d culture adherent monocytes in medium with 20 ng/ml IL-4 (5×10⁶ units/mg, Peprotech) and 50 ng/ml GM-CSF (1×10⁷ units/mg, Peprotech). Cytokines were added again on day 4 in RPMI-1640 medium supplemented with 10% FBS. Following this procedure more than 90% cells belonged to the immature DC phenotype (CD1a⁺, HLADRlow, CD14⁻, CD83⁻, CD86^{low}, CD80^{low}). On day 5 cells were treated with nothing or with recombinant human IFN-γ (1000 U/ml) for 18 h before stimulation with NadA (0.0375-5 μM) or LPS (1 μg/ml). After 24 h cells were harvested and analysed. Culture supernatants were collected frozen in liquid nitrogen and conserved at −80° C. for cytokine analysis.

For naïve Th cell purification, frozen aliquots of PBMC were thawed and depleted of memory CD45RO⁺ by magnetic depletion using antibody against CD45RO (Pharmingen), goat anti-Mouse IgG Microbeads (Milteny Biotech), LD separation columns (Milteny Biotech) and a VarioMACS magnet (Milteny Biotech) according to the manufacturer's instructions. CD45RO-cells were further incubated with human CD4 Microbeads (Milteny Biotech) for positive magnetic selection of highly pure T naïve helper cells with MS columns (Milteny Biotech) and a MiniMACS magnet (Milteny Biotech). T-cell fractions were >95% CD4⁺ CD45RA as assessed by flow cytometry. All cultures were performed in endotoxin-free RPMI-1640 (GIBCO BRL) supplemented with 10% heat inactivated FBS (Euroclone). All cells were kept at 37° C. in a humidified atmosphere containing 5% (v/v) CO₂, unless otherwise specified.

### Microscopy

DCs cultured for 5 days in 6-well plates (Costar) were treated with recombinant human IFN-γ (1000 U/ml) for 18 h before NadA (1.5 μM) or LPS (1 μg/ml) stimulation for 4 h. Control cultures were untreated cells or treated with IFN-γ alone.

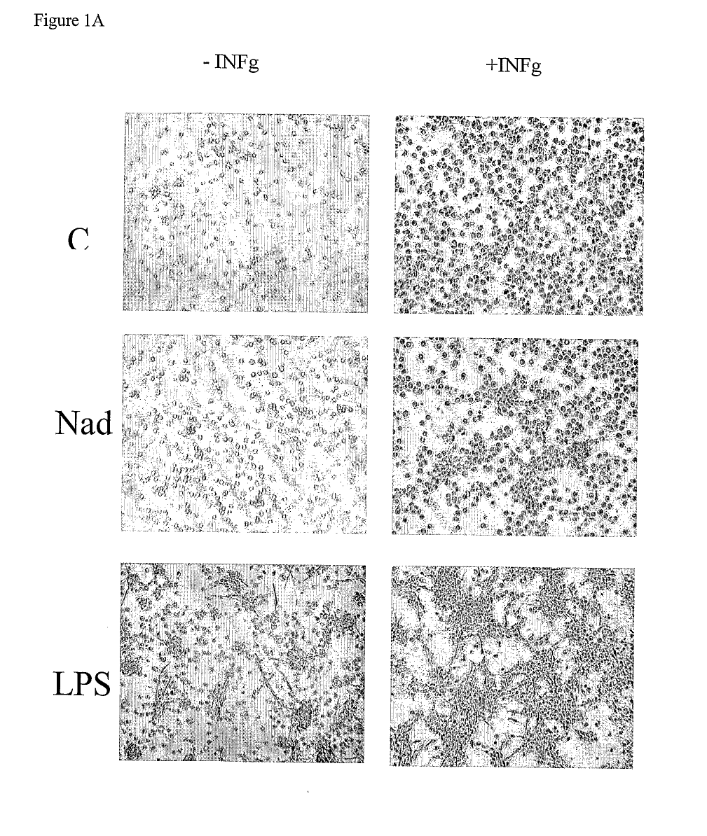

Alteration of cell morphology and distribution is a good indicator of DC activation. Analysis of the cells' morphology by optical microscopy suggested that NadAΔ351-405 (1.5 μM) activated immature mo-DCs, only when they were subjected to a priming (18 hours) with IFN-γ (1000 U/ml). In such case, after a short incubation (3 hours) with the meningococcal protein, some cells became elongated and tended to cluster, although less intensely than after stimulation by maximally active LPS (1 μg/ml) (FIG. 1A).

The same experiment was also carried out using macrophages. These showed reduced clustering following NadA treatment compared to LPS treatment (FIG. 1B).

To test the difference between the effect of recombinant soluble NadA and OMV expressed NadA, the experiments were repeated using OMV NadA. FIGS. 1C and 1D show that OMV_{NadA}_{^{−}} and OMV_{pET}_{^{−}} induce a comparable morphological effect on monocyte and macrophage cells, whereas treatment with OMV_{wt} and OMVₖₒ results in the cells becoming elongated and tending to cluster, although less intensely after co-stimulation with IFNγ. Thus, NadA induces both morphological and spacial changes that are more apparent with recombinant soluble NadA compared to OMV expressed NadA.

### Flow Cytometry Analysis

After differentiation, DC were routinely stained with phycoerytrin conjugated monoclonal antibodies to human CD14, CD1a, CD83, CD86 (B7.2), CD80 (B7.1), MHC II (HLA-DR), purchased from BD-Pharminghen and Caltag. In parallel, cells were stained with the isotype matched control mAb. Cells were immunostained with the proper dilution of PE-conjugated anti human monoclonal antibodies at 4° C. for 30 min in 100 μl of phosphate-buffered saline pH 7.2 (PBS, GIBCO BRL) containing 1% FBS and 0.1% NaN3 (FACS buffer). After washing, propidium iodide was added to exclude dead cells and cell fluorescence intensities of the gated populations were measured with a EPICS XL-MCL (Coulter) flow cytometer and analyzed with EXPO 32ADC XL 3COLOR or WinMDI 2.8. software. Data were collected on 10000-20000 events.

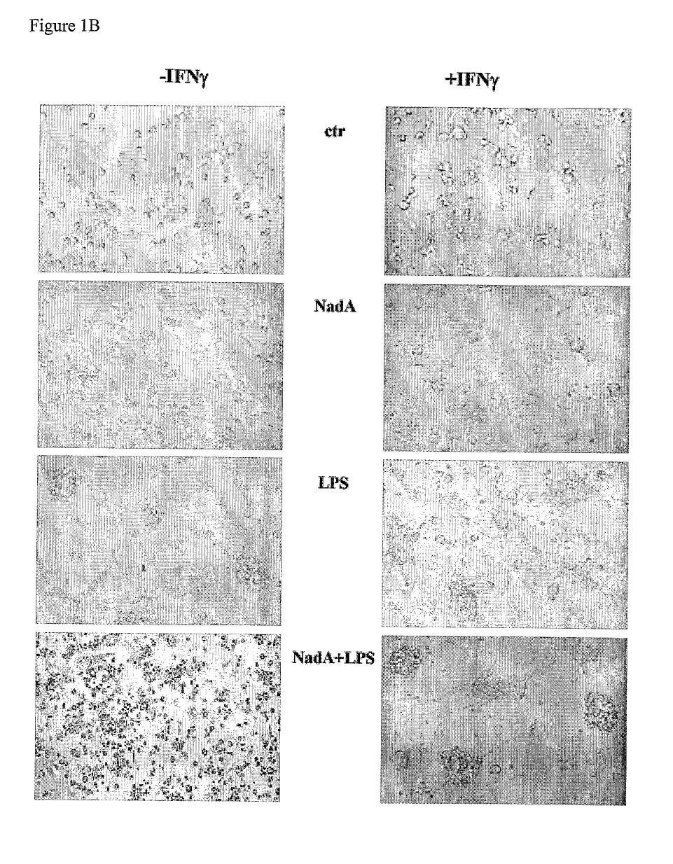

CD83 was not increased after a 24 hour exposure to NadAΔ351-405 (1 μM) (see FIG. 2). However, after IFN-γ priming, NadA stimulation boosted CD83 level to ˜50% of that induced by LPS. IFN-γ priming also influenced the expression of CD86, the co-receptor essential for MHC-II mediated antigen presentation. CD86 level in mo-DCs treated with NadA was greatly enhanced after IFN-γ priming and reached the same value observed in LPS-treated cells. IFN-γ priming scarcely affected LPS-induced expression of CD83 and CD86. The expression pattern of CD80, the other co-stimulatory molecule necessary to T lymphocyte activation, was almost superimposable to that of CD86 (not shown). Control plasma membrane HLA-DR, a marker of T-epitope presenting MHC-II proteins, already expressed in immature cells, was partially increased by NadA and roughly doubled by LPS. Although IFN-γ priming was per se sufficient to up-regulate surface HLA-DR, subsequent stimulation with NadA and LPS further increase such basal level, in a similar way.

### Cell Binding Experiments

In some cases, DCs primed or not with IFN-γ were treated at 37° C. for 1 hour with FCS/RPMI containing Bafilomycin A1 200 nM, incubated at 37° C. (in RPMI medium supplemented with 10% FBS and Bafilomycin A1) or 0° C. (in PBS supplemented with 10% FBS) for 3 hours with different concentrations (0.0375-5 μM) of Alexa-NadAΔ351-405 or NadA. Afterward cells were washed and suspended in FACS buffer for FACS analysis. Scatchard plots were constructed from data obtained from cell-associated mean fluorescence intensities due to cell-bound Alexa NadA were measured. The dissociation constant Kd and maximal binding capacities were then determined by Scatchard analyses.

Effect of NadA on mo-DC Maturation Markers

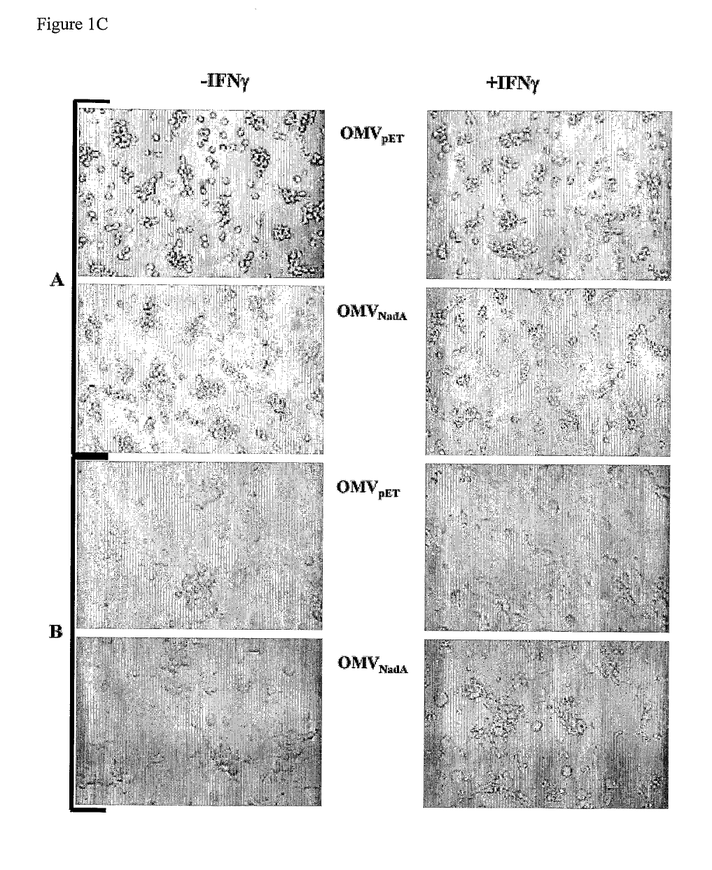

The effect of NadAΔ351-405 on mo-DCs was further investigated by measuring the expression of typical maturation markers (FIG. 3). CD83 was not increased after a 24 hour exposure to NadAΔ351-405 (1.5 μM). However, after IFN-γ priming, NadA stimulation boosted CD83 expression to ˜50% of the amount induced by LPS. IFN-γ priming also influenced the expression of CD86, the co-stimulatory molecule associated with dendritic cell maturation. CD86 expression on mo-DCs treated with NadA was greatly enhanced after IFN-γ priming and reached the same value observed in LPS-treated cells. IFN-γ priming alone scarcely affected LPS-induced expression of CD83 and CD86. The expression pattern of CD80, the other co-stimulatory molecule necessary for T lymphocyte activation, was very similar to that of CD86 (not shown). Control plasma membrane HLA-DR expression, a marker of T-epitope presenting MHC-II proteins, already expressed in immature cells, was partially increased by NadA and roughly doubled by LPS treatment. Although IFN-γ priming was per se sufficient to up-regulate surface HLA-DR expression, subsequent stimulation with NadA and LPS further increased the basal value in a similar way.

### Bio-Plex Multiplex Cytokine Assays

The antibody pairs used, directed against different non-competing epitopes of a given cytokine, were purchased from BioRad. Calibration curves from recombinant cytokine standard were prepared with four-fold dilution steps in RPMI-1640 medium containing 10% FBS. Assays were carried out in 96-well sterile pre-wetted filter plates at room temperature and protected from light. A mixture containing 5000 microspheres per cytokine was incubated together with standard or sample in a final volume of 50 μl for 30 min, under continuous shaking (300 rpm). After three washes by vacuum filtration with Bio-Plex washing buffer a cocktail of biotinylated antibodies diluted in Bio-Plex detection antibody diluent was added (25 μl to each well). After a 30 minutes incubation and washing, Streptavidin-PE diluted in Bio-Plex Assay buffer was added (50 μl per well). At the end of 10 minutes incubation under continuous shaking and after washing the fluorescence intensity of the beads was measured in a final volume of 125 μl of Bio-Plex assay buffer. Data analysis was done with Bio-Plex Manager software using a five-parametric-curve fitting. The detection limits were 0.2 μg/ml.

Measurements of surface maturation markers suggested that NadAΔ351-405 induces a mo-DC phenotype competent for antigen presentation, only after IFN-γ priming (see above and FIG. 3). To extend the characterisation of the functional properties of NadA-stimulated mo-DCs, we also investigated the production of local mediators, with or without IFN-γ priming. The secretion of inflammatory cytokines TNFα and IL-6, of chemokine IL-8 and of the regulatory cytokines IL12p70 and IL-10 was measured with a Bio-Plex suspension array in the extracellular media from mo-DCs stimulated for 24 hours. NadAΔ351-405 (1 μM) induced a significant production of TNFα and IL-6, which was increased by IFN-γ priming to ˜24% of maximal LPS production. IL-8 secretion, measurable also in non stimulated cells, was further increased by NadAΔ351-405 in the absence of priming. In contrast with what seen for TNFα and IL-6 secretion, IFN-γ priming slightly inhibited NadA-induced IL-8 secretion, which was in both cases ˜24% of that induced by LPS. Under no condition in this example was NadA able to induce IL-10 production.

IL-12p70 production by NadA-stimulated mo-DCs, undetectable as in control cells, became significant after IFN-γ priming. It is to be noted, however, that such IL-12p70 secretion level was low compared to the one induced by LPS (<2%). IL12-p40, the subunit that assembles with IL12-p35 to form biologically active IL12-p70, was detectable in the extracellular medium from NadA-treated cells and its level was further increased by IFN-γ priming. Also in this case maximal secretion was ˜2% of that induced by LPS.

IL12(p40) ELISA

IL12(p40) was measured by capture enzyme-linked immunosorbent assay (ELISA) with antibody pairs and cytokine standard purchased from Bender MedSystems. The concentrations of IL12(p40) in the cell-free supernatants were determined with ELISA kits according to the manufacturer's instructions. The detection limit of the assays was 20 μg/ml.

### Real-Time PCR Analysis

Mo-DC were pre-treated or not with IFN-γ 1000 U/ml and stimulated with NadA 1.5 mM and LPS 1 mg/ml for 3-5-8 h. Treated and untreated cells were pelleted and used for RNA isolation. Total RNA was extracted using the TRIzolÒ reagent (GibcoBRL) according to the manufacturer's instruction, precipitated and resuspended in 6-8 ml of RNAse free water (Gibco). RNA was quantified with a fluorescence spectrophotometer (BeckmanDU 530). First strand cDNA was prepared from 4 mg of total RNA by using the Superscript™ II Reverse Transcriptase (Invitrogen) with oligodT primers (Sigma Genosys). The cDNA levels of IL12p35, IL12p40, IL-23p19, TNF-α and IL-6 were quantified by Real Time quantitative PCR using a qPCRTM Core Kit for Sybr Green I (Eurogentec) with a GeneAmp 5700 Sequence Detection System according to the manifacturer's instructions (Applied Biosystems). After an initial denaturation step at 95° C. for 10 min, temperature cycling was initiated. Each cycle consisted of 30 sec at 95° C. and 30 sec at 60° C. (TNF-α at 61° C. and p19 at 63° C.); in total 40 cycles were performed. The following primers were used:

| | |
|---|---|
| IL12p35 sense | |
| 5′-ATGGCCCTGTGCCTTAGTAGT-3′, | (SEQ ID NO: 6) |
| IL-12p35 antisense | |
| 5′-CGGTTCTTCAAGGGAGGATTTT-3′; | (SEQ ID NO: 7) |
| IL-12p40 sense | |
| 5′-ACAAAGGAGGCGAGGTTCTAA-3′, | (SEQ ID NO: 8) |
| IL-12p40 antisense | |
| 5′-CCCTTGGGGGTCAGAAGAG-3′; | (SEQ ID NO: 9) |
| IL-23p19 sense | |
| 5′-TCCACCAGGGTCTGATTTTT-3′, | (SEQ ID NO: 10) |
| IL-23p19 antisense | |
| 5′-TTGAAGCGGAGAAGGAGACG-3′; | (SEQ ID NO: 11) |
| TNF-α sense | |
| 5′-ATGAGCACTGAAAGCATGATCC-3′, | (SEQ ID NO: 12) |
| TNF-α antisense | |
| 5′-GAGGGCTGATTAGAGAGAGGTC-3′; | (SEQ ID NO: 13) |
| IL-6 sense | |
| 5′-AACCTGAACCTTCCAAAGATGG-3′, | (SEQ ID NO: 14) |
| IL-6 antisense | |
| 5′-TCTGGCTTGTTCCTCACTACT-3′; | (SEQ ID NO: 15) |
| HMBS sense | |
| 5′-GGCAATGCGGCTGCAA-3′, | (SEQ ID NO: 16) |
| HMBS antisense | |
| 5′-GGGTACCCACGCGAATCAC-3′ | (SEQ ID NO: 17) |

All amplification products were cloned into a TOPO TA vector (Invitrogen) and quantified by Beckman DU 530 spectrophotometer. To obtain standard curves, samples from minipreps were serially diluted to concentrations ranging from 0.5×10⁻² to 0.5×10⁻⁷ fmol/ml. Amplified products (20 ml) together with a DNA ladder (Invitrogen) as a size standard were resolved on a 2% agarose in the presence of ethidium bromide.

The cDNA levels during the linear phase of amplification were normalized against HMBS. Each run was completed with a melting curve analysis to confirm the specificity of amplification and lack of primer dimers. CT values were determined by the GeneAmp 5700 SDS software using fluorescence threshold manually set and exported into Excel for analysis.

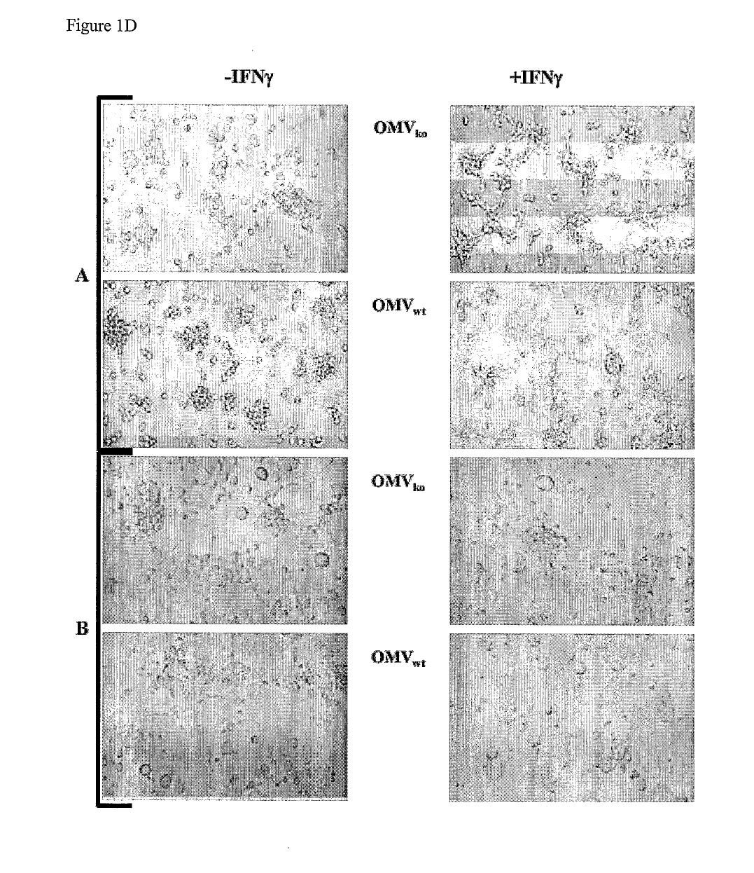

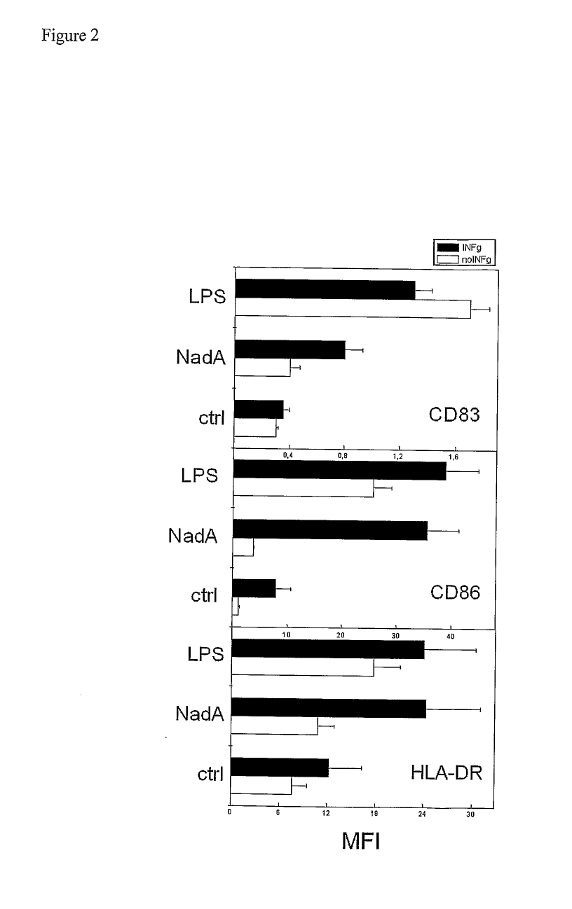

Data confirmed that IFN-γ priming augmented NadA-induced transcription of TNFα and IL-6 genes (FIG. 4). The levels of IL-12p40, IL-12p35 and of IL-23p19 transcripts were quantified, with the goal of gaining information on the transcription of the subunits forming IL-12p70, but also IL-23, which is composed of p40 and p19. IL-23, recently discovered, has an activity overlapping, although not completely, with that of IL-12. Results showed that IL12-p40, IL12-p35 and IL-23p19 transcriptions were all increased by NadA only if cells were primed with IFN-γ. The transcription activities of genes encoding for IL-6, TNFα, p40, p35 and p19 induced by NadAΔ351-405 in IFN-γ primed mo-DCs could be estimated to be <1% of the one observed in LPS-activated cells.

### Allogenic Mixed Leukocyte Reaction and Naive CD4+ T-Cell Proliferation

Allogenic mixed leukocyte reaction was performed with irradiated (3000 rads from a ¹³⁷Cs source) mo-DC and purified allogenic T cells. Graded numbers of DC cultured for 18-24 h with NadA 1.5 μM, LPS 1 μg/ml (positive control) and non stimulated DC (negative control) pretreated or not with IFN-γ 1000 U/ml were washed and cultured with allogenic CD4+ naïve T lymphocyte (0.3×10⁵ cells/well) for 5 days at 37° C. in a humidified CO₂ incubator in round-bottom 96-well microtiter plates (Costar). Proliferation was measured by pulse-labelling triplicate wells for 6 h with 1 μCi of 3H-Thymidine/well (Amersham Biosciences). Negative controls included T naive cells incubated or DC incubated alone.

³H-Thymidine incorporation was measured by harvesting cells onto glass fiber filter paper (Pall Corporation, Life Sciences) using a 96-well semiautomatic cell harvester (Multiwash 2000, Dynatech) and counting by liquid scintillation in a O-counter (Wallac 1409 liquid scintillation counter).

### Intracellular Detection of IFN-γ and IL-4 by Flow Cytometry

To provide additional evidence on the specificity of NadA effects on mo-DCs, we decided to prove and characterise its physical interaction with the putative target cells. A clear binding of Alexa-labelled NadAΔ351-405 to mo-DCs, not significantly altered by IFN-γ priming, was measured at 37° C. by flow-cytofluorimetry.

Mo-DC pre-treated in various conditions were co-cultured with naïve T cells for five days and re-stimulated with ionomycin 1 μg/ml and PMA 10 ng/ml for 2.5 h and for 3 h in the presence of a Brefeldin-A, (10 μg/ml final concentration). Cells were then washed and fixed for 15 min (Fix and Penn cell permeabilization kit, Caltag). After one washing step cells were permeabilised and stained with FITC conjugated anti interferon-γ mAB (BD-Pharmigen) and PE conjugated anti IL-4 mAb (Caltag) or with irrelevant isotype control for 30 min. Then cells were washed again, resuspended and analysed by flow cytoflorimetry.

Data showed that, independently on IFN-γ priming, significant cell association of NadA was evident in the submicromolar concentration range, but did not reach a complete saturation at concentrations up to 5 Scatchard plot analysis showed that the majority of binding sites (70-80%) associates to NadA with a low affinity (3-5 μM), while a minor fraction of binding sites (20-30%) had an apparent Kd around 50-100 nM. The existence of two kinds of binding sites on mo-DCs was confirmed at 0° C., a condition that eliminates endocytosis, although in this case the binding capacity was quite reduced. Competition experiments with non-labelled NadAΔ351-405 (up to 5 μM) confirmed the presence of specific NadA receptors on mo-DCs, although the analysis at higher ligand concentration was precluded by material limitation. The analysis of fluorescence distribution due to Alexa-NadAΔ351-405 binding at submicromolar and micromolar concentrations suggested that high affinity sites are present in very variable amounts within the cellular population, while low affinity ones are more homogeneously expressed. All together these experiments demonstrate the existence of high and low affinity binding sites for NadA on immature mo-DCs. They as well exclude that the synergic effect of IFN-γ priming results from an increased association of NadA to mo-DCs.

Without being limited to a particular hypothesis, it can be speculated that the response of mo-DCs to NadA can be modulated by several factors. First condition for mo-DCs reaction is the pre-existence of INF-g in the tissue for a prolonged time, a condition that may be achieved, e.g., by an inflammation state. In other words, the mere presence of NadA on DCs has little meaning for the immune system, unless other PAMP signal an infection. Given the presence of other microbial stimuli in the tissue, mo-DCs become able to sense the presence of low amount of adhesin bound to high affinity receptors, and respond by up-regulating the antigen presenting machinery and by secreting few IL-12, allowing the initiation of T cell proliferation and of an immune response. In case adhesin concentration is higher, mo-DCs not only may further boost their antigen presenting efficacy, but may as well participate in the amplification of the inflammatory reaction. In can be speculated that the first reaction occurs when infection of meningococcal cells is at the beginning, or sub-clinical: in this case mo-DCs functional, response is only aimed at triggering an immune response, without exacerbating the inflammatory reaction. However when meningococcal infection is more intense, and NadA more concentrated, occupation of mo-DCs low affinity sites may not only result in a further increase of APC functions but also in a controlled secretion of proinflammatory cytokine, involving mo-DCs in the amplification of local defence mechanism necessary to counteract the bacterial invasion.

Dose Response of mo-DC Activation by NadAΔ351-405

The dose response effect of NadAΔ351-405 on CD86 overexpression in mo-DCs and on their cytokine secretion was analysed and compared with cell binding. With no IFN-γ priming, CD86 was not different from control cells below 1 μM NadA, but increased almost linearly at higher concentrations. On the contrary, after IFN-γ priming, NadA effectively induced CD86 also in the submicromolar concentration range. Comparison with the NadA binding curve in the same conditions, showed that CD86 induction correlated with occupation of low affinity sites in non-primed cells, while of both high and low affinity sites in primed ones. It is to be however emphasised that IFN-γ potentiation of NadA effect was strong at low concentrations (from no effect to a sensible one), while minor at higher concentrations (a relative 2-3 fold increase). Analysis of the cellular distribution of CD86 expression revealed that, after IFN-γ priming, a fraction of mo-DCs was very responsive to NadA at concentrations corresponding to the occupation of high affinity binding sites.

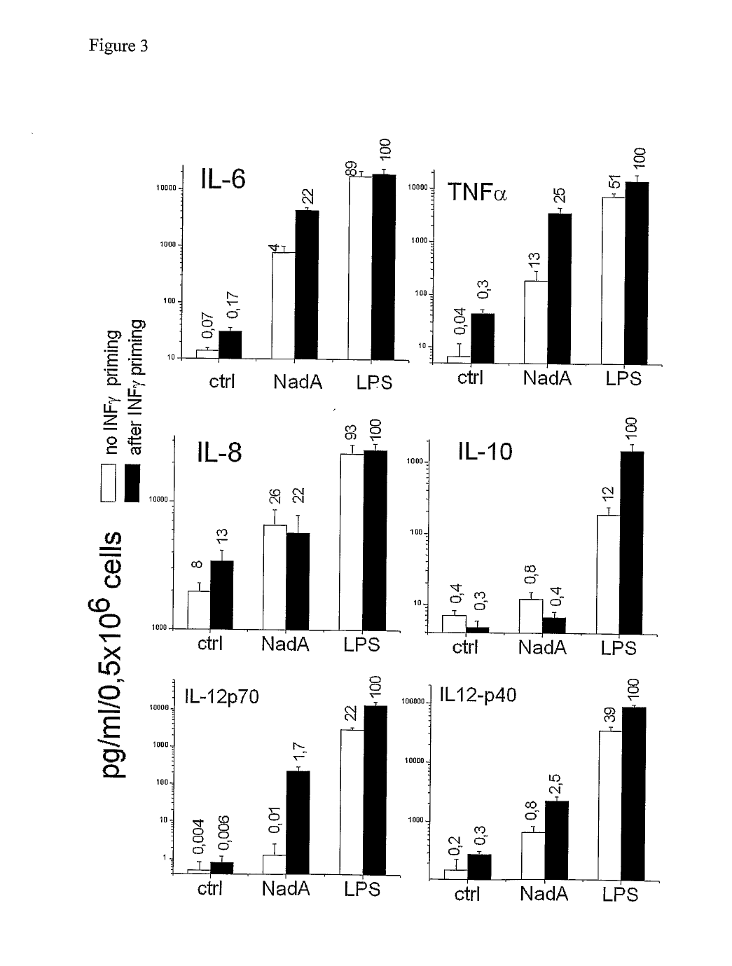

In parallel, we measured cytokine secretion in the extracellular medium, using a Bio-Plex suspension array. IL-6, TNFα and IL-8 were evident in samples from non-primed cells treated with NadAΔ351-405 only at concentrations higher than 1 μM. After IFN-γ priming, very low quantities of IL-6, TNFα and IL-8 were evident below 1 μM NadA. On the contrary, IFN-γ priming potentiated IL-6 and TNFα secretion, while partially inhibited IL-8 production, at concentrations higher than 1 μM. IL-12p70, undetected until up to 5 μM NadA, in the absence of IFN-γ priming, became evident and reached a plateau below 1 μM NadA, after IFN-γ priming. IL-10 secretion was undetectable until up to 5 μM NadA, without or with IFN-γ priming (FIG. 6).

### T Lymphocyte Proliferation and Differentiation

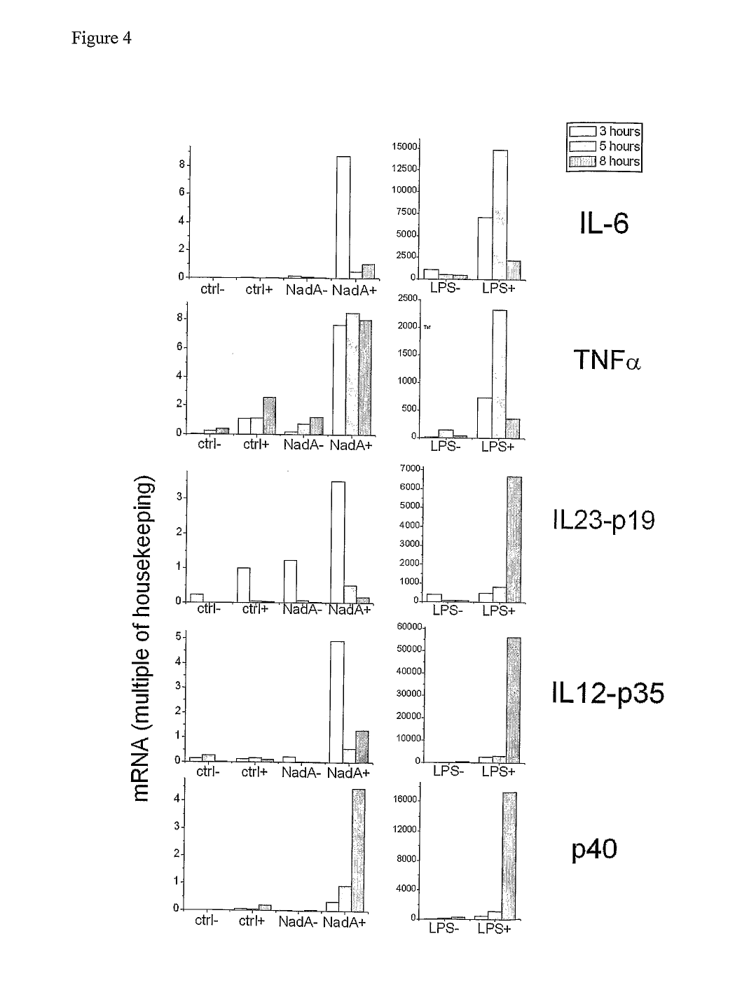

Mixed Lymphocyte Reaction experiments performed with isolated allogenic naive TCD4⁺ cells, showed that NadAΔ351-405 (1 μM), in the absence of IFN-γ priming, was not able to induce a mo-DC phenotype competent for T lymphocyte activation. On the contrary, mo-DCs primed with INFγ and then stimulated with NadA induced a significant T cell proliferation, which was 30-40% of the one supported by LPS-matured mo-DCs. IFN-γ priming did not increase the ability of LPS-matured mo-DCs to activate T lymphocytes (FIG. 7A).

The differentiation of T lymphocytes activated by IFN-γ primed NadA- or LPS-matured mo-DCs, determined by measuring intracellular IFN-γ and IL-4, is shown in FIGS. 7 B and C, as representative of one out of the three different DC concentrations and of one out of the two donors tested, quantified ranking the cells in INFγ⁺, IL-4⁺ and IFN-γ⁺/IL-4⁺ and expressing the data as % of the total T cell population. After IFN-γ priming, LPS activated mo-DCs, strongly polarised T cells towards the IFN-γ+phenotype (36-65%), while IFN-γ⁺/IL-4⁺ and IL-4⁺ cells were few. Within T cells induced by NadA-matured mo-DCs, the IFN-γ+ phenotype, although still predominant (13-31%), was as well associated with a significant fraction of IFN-γ⁺/IL-4⁺ (4-12%) and IL-4⁺ (3-18%) cells.

### Prediction of Heparin-Binding Domains

Using the heparin-binding motifs proposed by Cardin and Weintraub, (XBBXBX and XBBBXXBX, where B is a basic amino acid and X any other amino acids), potential heparin-binding domains were identified in NadA.

Chang cells were incubated at 37° C. for 3 h with NadA 600 nM and re-incubated 10 min at 37° C. with heparin. A dose-dependent reduction in protein binding to Chang cells in the presence of heparin was observed using fluorescence microscopy.

Using, affinity chromatography with different buffers (phosphate 20 mM and hepes 20 mM), pH (5.5, 7.4 and 8.0) and CaCl₂ concentrations, the binding of NadA to heparin was further investigated. A solution of hepes 20 mM containing 5 μg NadA was added to 100 μl of heparin-agarose pre-equilibrated in the same buffer. After washing, bound protein was eluted using a salt gradient (0.05-3M NaCl). All the fractions from the previous steps were collected and transferred to nitrocellulose membrane using Dot-blot. The protein was detected with a rabbit polyclonal anti-NadA antibody and phosphatase alcalyne-conjugated goat anti-rabbit anti-IgG with its substrate. This protocol has shown a heterogeneous behaviour for NadA. A fraction of the protein elutes at physiological salt concentrations (100-150 mM NaCl) and a further one elutes at high salt concentrations (up to 3M NaCl).

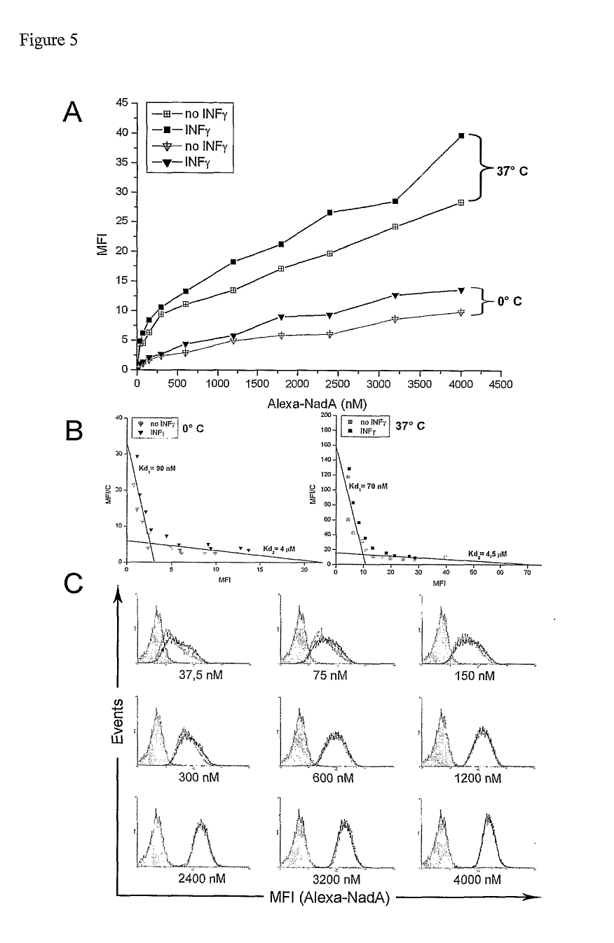

Expression of full length NadA on the outer membrane increases the adhesion of an E. coli model to the human conjunctival cell line Chang [73]. Consistently, soluble isolated NadAΔ351-504 has been shown to bind to Chang cells [71]. Data shown in FIG. 8 demonstrate that binding of Alexa-labelled NadAΔ351-504 to Chang cells is competed by non-labelled NadAΔ351-504 in a dose dependent manner. Signal decrease indicates a low-affinity interaction with specific receptors, compatible with the binding curve reported in reference 35. The specificity of NadA displacement in Chang cells is confirmed by experiments conducted with CHO-K1 cells, which show a significant association of Alexa labelled NadAΔ351-504. However, this binding is not modified by non-labelled NadAΔ351-504. Similar experiments performed with mo-DCs demonstrated the existence of a specific binding to these cells, but not to other leukocytes like PMNs. As in Chang cells, Alexa-NadAΔ351-504 binding to mo-DCs is competed by non-labelled NadAΔ351-504, suggesting the existence of similar receptors able to specifically associate with NadA at low affinity. Therefore, the binding of NadA to PMNs appears to be non-specific. However, NadA binds to specific receptors present on both Chang epithelial cells and mo-DCs.

### Interaction Between NadA and the Complement System

Confocal microscopy analysis has shown that NadA clusters on the bacterial surface and masks the binding of E. coli specific antibodies.

Complement activation by the classical pathway was investigated. Bactericidal assays were performed with a human serum pool (NHS). The susceptibility to complement-mediated lysis was determined after a 30 min incubation, using a E. coli BL21 strain transformed with pET21b plasmid bearing allele 3 of full-length NadA gene (E. coli-NadA) and a control, carrying the pET21b plasmid with no insert (E. coli-pET). The number of surviving bacterial cells was measured by serial agar plating and colony counting. No significant difference was noted between the two strains.

Complement activation by the alternative pathway was also investigated. Bactericidal assays were performed with NHS in the presence of 2 mM Mg²⁺ and 10 mM EGTA, a calcium chelator that specifically inhibits the classical pathway activation. The susceptibility to complement-mediated lysis was determined by incubating E. coli-NadA and E. coli-pET with 0-75% NHS at 37° C. for 15 min under agitation. The number of surviving bacterial cells was measured by serial agar plating and colony counting. The results showed a significant decrease in killing effect of alternative pathway in the E. coli-NadA strain. These data suggest that NadA may specifically interfere with the activation of the alternative pathway in the complement system.

Moreover, the effect shown is human specific: in guinea pig, rat and mouse sera the presence of NadA on the bacterial cells did not inhibit the alternative pathway at any of the serum concentrations tested.

To investigate the interaction of NadA with immune system soluble factors, an analysis of C3 and factorB deposition on the bacterial surface was performed using SDS-PAGE and Western blotting. Assays were performed with C9 defective human serum in the presence of 2 mM Mg²⁺ and 10 mM EGTA. Bacterial cells were subjected to SDS-PAGE and Western blot analysis, performed with specific anti C3 or FB antibodies directly or after hydroxylamine treatment. The results showed an increase in C3 and FB fragment deposition on the control strain surface.

Furthermore, a soluble recombinant form of NadA (NadAΔ351-405) has been found to partially inhibit the alternative pathway when added at 3 μM concentration in the bactericidal assay performed with the control E. coli-pET.

Comparison of NadA Effect on mo-DCs with Other Common PAMP Stimuli

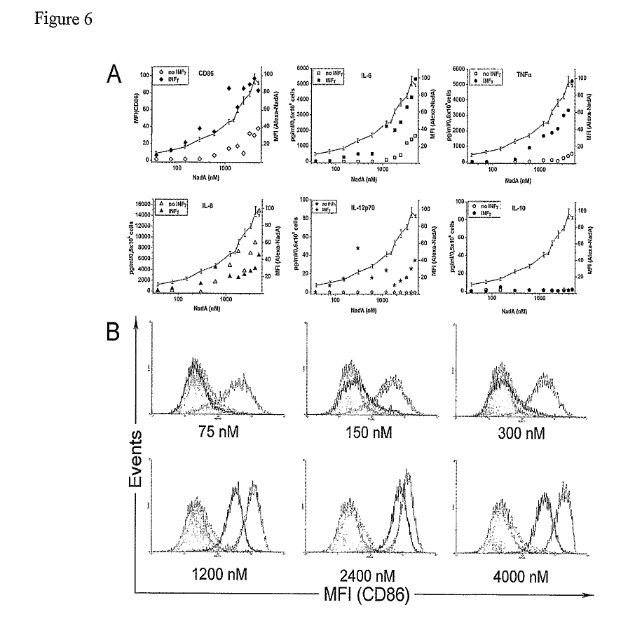

The effect of NadA on mo-DCs was compared with the action of known classical PAMP stimuli, typical of Gram-ve bacteria: flagellin, non-methylated DNA and LPS. FIG. 9A shows that administration of flagellin at a high dose (10 μg/ml) results in a significant increase of CD86 expression, which is further enhanced by IFN-γ priming to a value comparable to that induced by NadA 1.5 μM. CpG, a ligand resembling non-methylated bacterial DNA, is ineffective in induction of CD86 expression at concentrations up to 10 μg/ml, even after IFN-γ priming. LPS up to 0.1 ng/ml had no effect in the absence of IFN-γ priming and only a slight one after priming. Maximal stimulation with LPS (0.1 μg/ml) resulted in a strong effect without IFN-γ, which was doubled by IFN-γ priming. Some IL-12(p70) secretion, comparable to that induced by both 0.25 μM (9 μg/ml) and 1.5 μM (50 μg/ml) NadA, was observed with a high flagellin dose (10 μg/ml), after INF-γ priming. CpG at high doses (10 μg/ml) had an even weaker effect and LPS up to 100 pg/ml was ineffective. Maximal LPS stimulation resulted in a much higher secretion of IL12(p70) after IFN-γ priming (FIG. 9B). These data exclude that the effect seen with the NadA preparations is due to contamination by non-methylated bacterial DNA, which can be estimated to be <36 pg/ml (NadA 1.5 μM) in the assay. In addition, they exclude the fact that LPS, measured to be <18 pg/ml (1.5 μM NadA) in the assay, is responsible for NadA preparation activity, since even after IFN-γ priming both CD86 and IL-12(p70) were poorly or not increased by LPS up to 100 pg/ml.

Contamination by flagellin, although this protein induces CD86 and IL-12 in a way which recalls NadA, is very unlikely to account for NadA preparation activity. In fact, since 10 μg/ml flagellin shows the same effect on IL-12 secretion as 0.25 μM NadA which corresponds to 9 μg/ml, this implies flagellin contamination comparable to the amount of the purified protein. However, this possibility is excluded by SDS-PAGE, western blot and HPLC analysis, that failed to detect a band corresponding to flagellin in the preparation.

R-848 Co-Stimulation Enhances IL-12p70 Secretion by NadA Treated mo-DCs

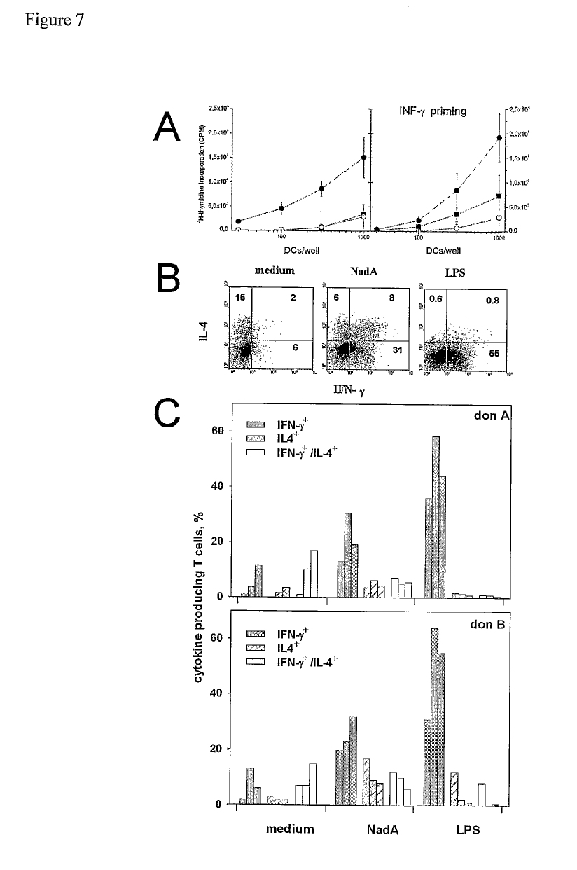

The antiviral drug R-848 is known to synergise the action of some PAMPs in inflammatory cells and DCs. This is believed to be due to the mimicking by this drug of free bacterial RNA. We therefore investigated the potentiating effect of R-848 on NadA and on other bacterial stimuli with or without IFN-γ priming. With no IFN-γ priming, R-848 alone (1 μM) produced a weak increase in CD86 expression in mo-DCs (FIG. 10). Co-stimulation with NadA (1.5 μM) resulted in an addition of the two effects, a situation which is also seen following co-stimulation with R-848 and flagellin (10 μg/ml). LPS 0.1 ng/ml showed no effect even with R-848 co-stimulation, and R-848 did not increase the strong effect of 0.1 μg/ml LPS. After IFN-γ priming, R-848 stimulation resulted in an increased of control CD86 level, reaching an intense value, which corresponded to about half of the maximal value induced by LPS. Again, co-stimulation with NadA 1.5 μM appeared to result in a sum of the two separate effects, leading to maximal CD86 expression. In the case of flagellin, and of LPS 0.1 μg/ml, a high level of CD86 expression was observed after IFN-γ priming, which was not further increased by co-stimulation with R-848. LPS 100 μg/ml had no effect even after co-stimulation with R-848.

The analysis of IL12(p70) secretion by mo-DCs treated in the same conditions revealed a specific behavior of NadA, with respect to flagellin and LPS.

Flagellin and LPS 100 μg/ml, were ineffective in inducing IL-12 secretion even with R-848 co-stimulation, even after IFN-γ priming. On the contrary mo-DCs co-stimulated with NadA and R-848 released a high level of IL-12 (2 ng/ml), a value which was increased 20 fold (45 ng/ml) after IFN-γ priming. A high dose of LPS (0.1 μg/ml) was very effective when administered to cells with R-848 in both priming and non-priming conditions, but a significant activity was seen even without R-848 co-stimulation (0.2 ng/ml with no priming and 6 ng/ml with priming). These data further exclude flagellin, bacterial DNA and LPS contaminations as the cause of the observed activity of NadA preparations, and prove that NadA effects are strongly synergized by R-848.

Interaction of NadA_{Δ351-405} with Human Monocytes

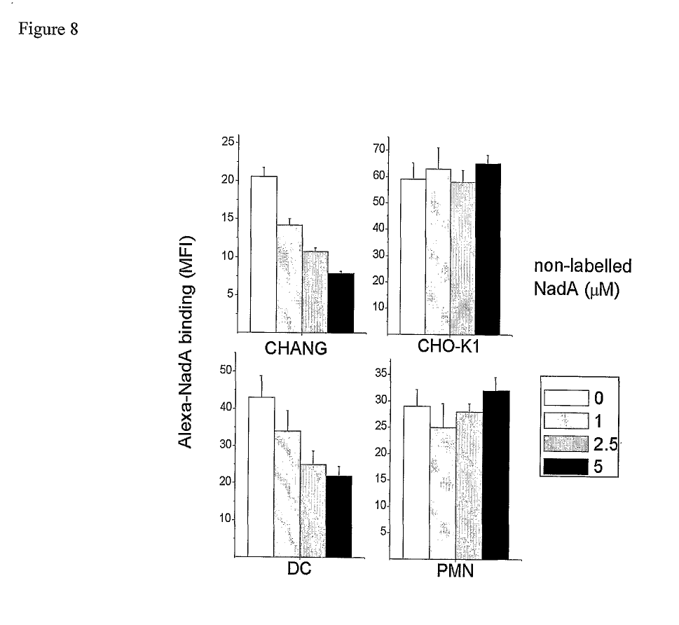

Alexa-NadA_{Δ351-405} staining, in the presence of BafA1 to block degradation of endocytosed ligand, followed by flow-cytofluorimetry was used to search for specific leukocyte targets of NadA. Results showed that a sub-population corresponding to ˜4% of leukocytes was positive for Alexa-NadA_{Δ351-405} staining. Double labelling experiments with CD-specific antibodies showed that these cells largely correspond to CD14-positive monocytes. Only small, or negligible, fractions of T lymphocytes (CD3 positive), B lymphocytes (CD19 positive) and NK cells (CD 16 positive) were alexa-NadA positive (FIG. 11).

The distributions of NadA associated to adherent monocytes cells was characterised by direct epifluorescence of living cells, or by confocal microscopy of fixed cells, following indirect immune staining with specific antibodies. NadA_{Δ351-405} was shown to be clustered in the monocytes plasma membrane and localized in intracellular vescicles.

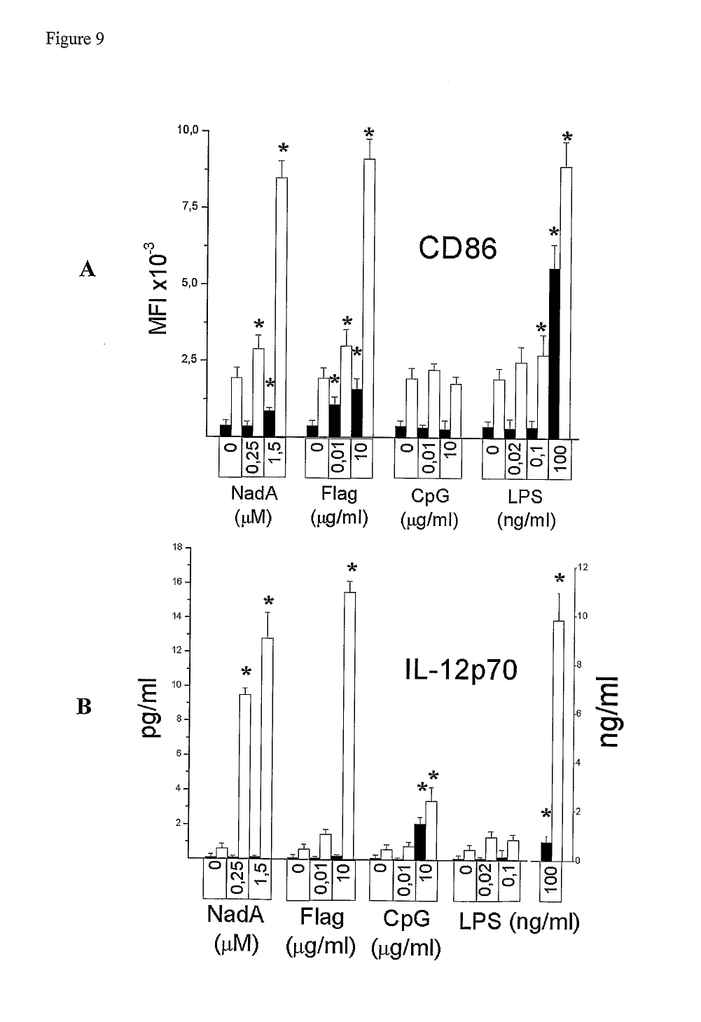

The dose-dependent preferential association of NadA to CD14⁺ monocytes was also confirmed by MFI analysis. Competition using non-labelled ligand ascertained whether NadA binding was specific (FIG. 12). In the presence of BafA1, a large excess of NAdAΔ351-405 (5 uM) resulted in a partial but significant decrease (−50%) of the signal associated to monocytes after incubation at 37° C. with 125 nM Alexa-NadA, revealing the presence of specific binding sites on monocytes. These data were confirmed using a NadA-I¹²⁵ conjugate and Scatchard Plot analysis and revealed that adhesin association to monocyte has an affinity (Kd) of ˜3 μM. Based on the molecular weight of the NadA monomer, this value is in fact ˜1 μM, since the recombinant protein used in the experiment is a homo-trimer.

Monocytes demonstrate Chang-like receptors and this suggest that the adhesin may be involved not only in mucosal colonisation and invasion, but also in tissue and blood invasion.

Interaction of NadA_{Δ351-405} with Human Macrophages

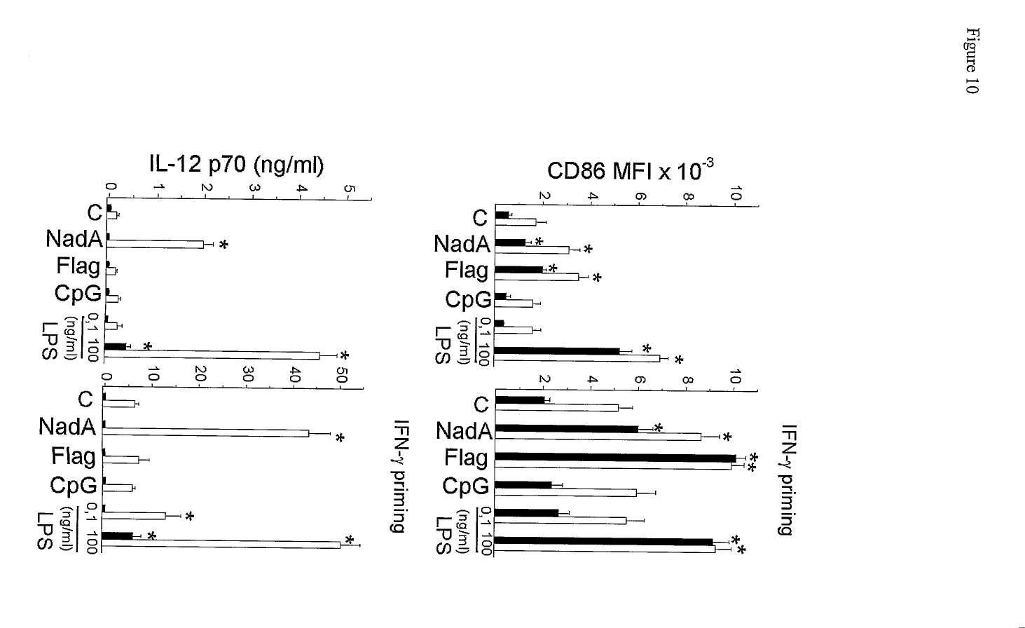

NadA binding to monocyte-derived macrophages was also investigated. A dose-dependent association of NadA to macrophages was confirmed by MFI analysis and competition by non-labelled ligand was used to ascertain whether NadA binding was specific. The results showed that NadA-specific binding sites on macrophage was detectable at 37° C. and there was a partial but significant decrease (−50%) the signal associated to cell (FIG. 13).

The distributions of NadA associated to adherent macrophage cells were characterised by direct epifluorescence of living cells, or by confocal microscopy of fixed cells, following indirect immune staining with specific antibodies. NadA_{Δ351-405} signal was localized in intracellular vesicles, mostly found in the perinuclear area.

### Phenotypical Analysis of NadA_{Δ351-405}-Treated Human Monocytes and Macrophages

The functional effect of NadA on human monocytes and macrophages was investigated using a soluble recombinant mutant lacking the membrane anchor and a full length protein expressed in E. coli OMV or N meningitidis OMV. To better define the immuno-modulatory activity of NadA, the cells were stimulated with protein plus or minus both microbial stimulus (LPS) and immunological stimulus (IFNγ).

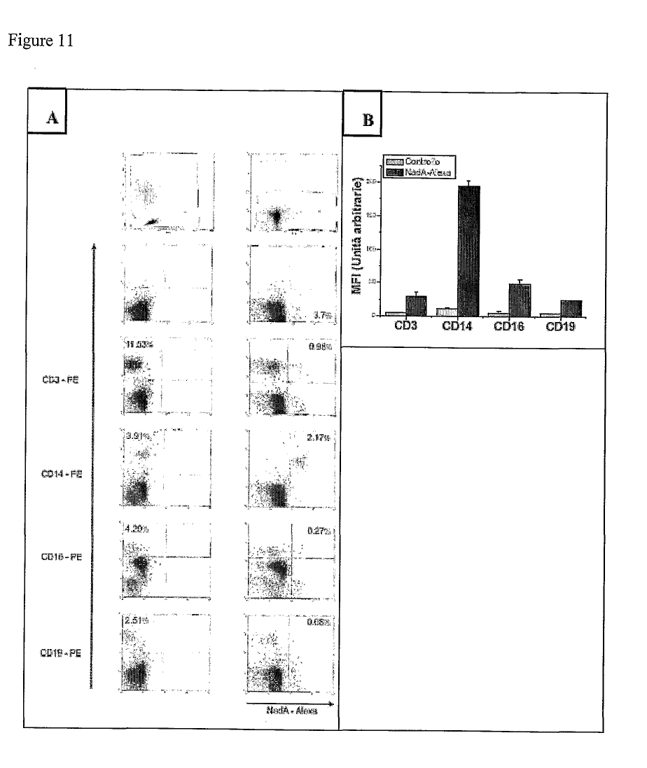

Western blot analysis was performed to investigate NadA expression in E. coli OMV. Results showed that the protein was found only on NadA⁺-E. coli mutant strains (FIG. 14).

### Analysis of Human Monocytes and Macrophages Surface Markers

The effect of NadA_{Δ351-405} on monocyte and macrophage cells was further investigated by measuring the expression of the antigen presentation marker MHC-II, the co-stimulatory molecules CD80 and CD86 and the cell adhesion molecule ICAM-1.

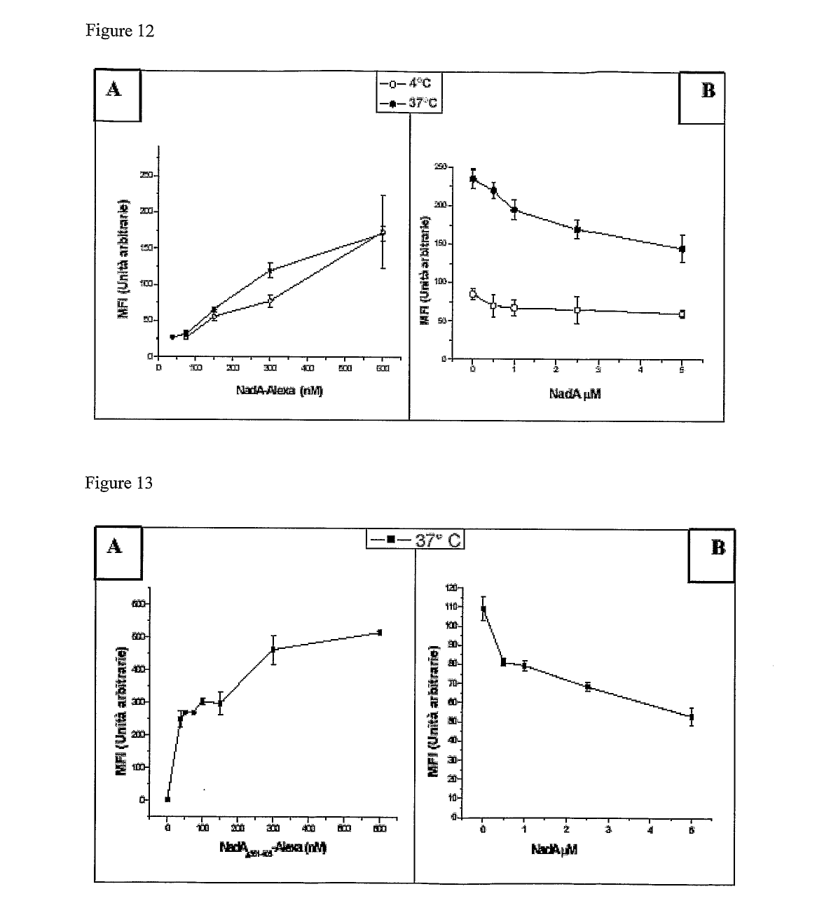

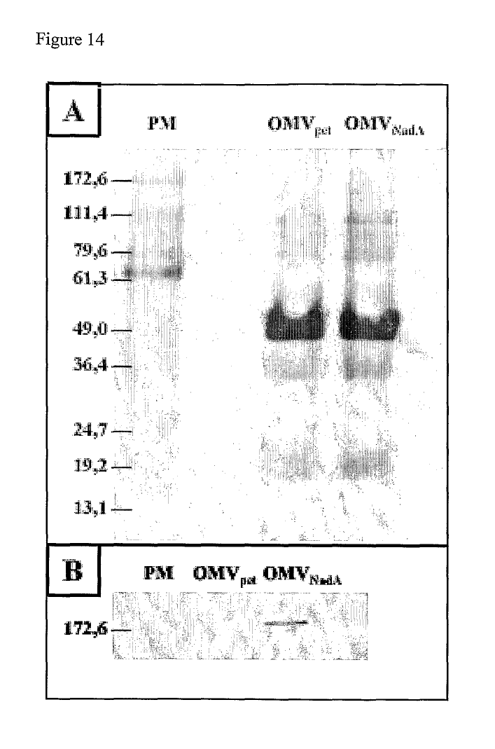

CD80 expression was increased after co-stimulation with NadA_{Δ351-405} and IFN-γ in both cellular models. No NadA immunomodulatory effect on CD86 or HLA-DR expression in monocytes was observed when the protein was used with LPS or IFN-γ (FIG. 15). Partial stimulation of CD86 expression by NadA_{Δ351-405} was seen in macrophages upon co-stimulation with LPS. The expression of HLA-DR in macrophages treated with NadA_{Δ351-405} was greatly enhanced after IFN-γ co-stimulation. ICAM-1 expression in macrophages was increased after exposure to NadA_{Δ351-405} (FIG. 16).

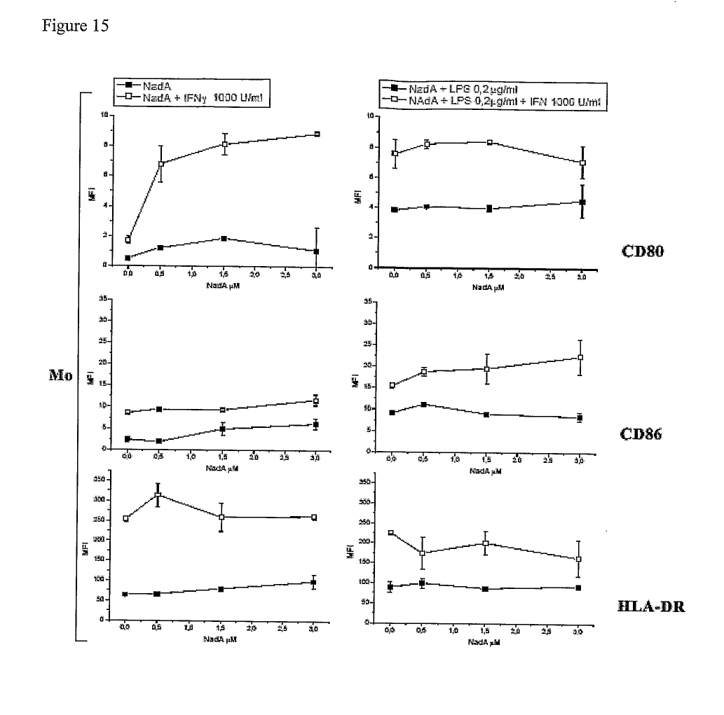

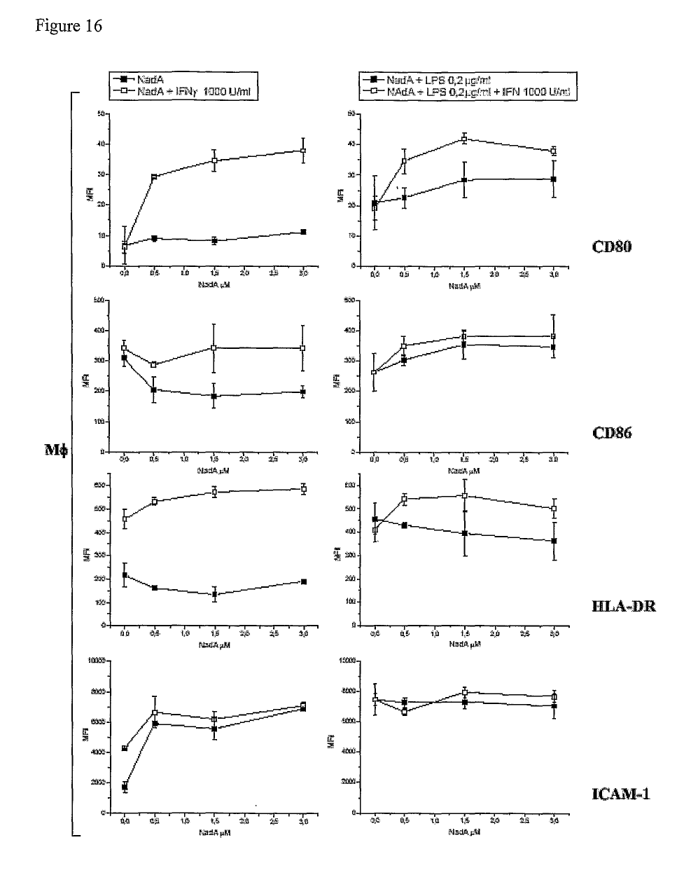

The expression profile of the various markers in both cellular models following stimulation with E. coli OMV with or without IFN-γ co-stimulation was similar (FIGS. 17 and 18).

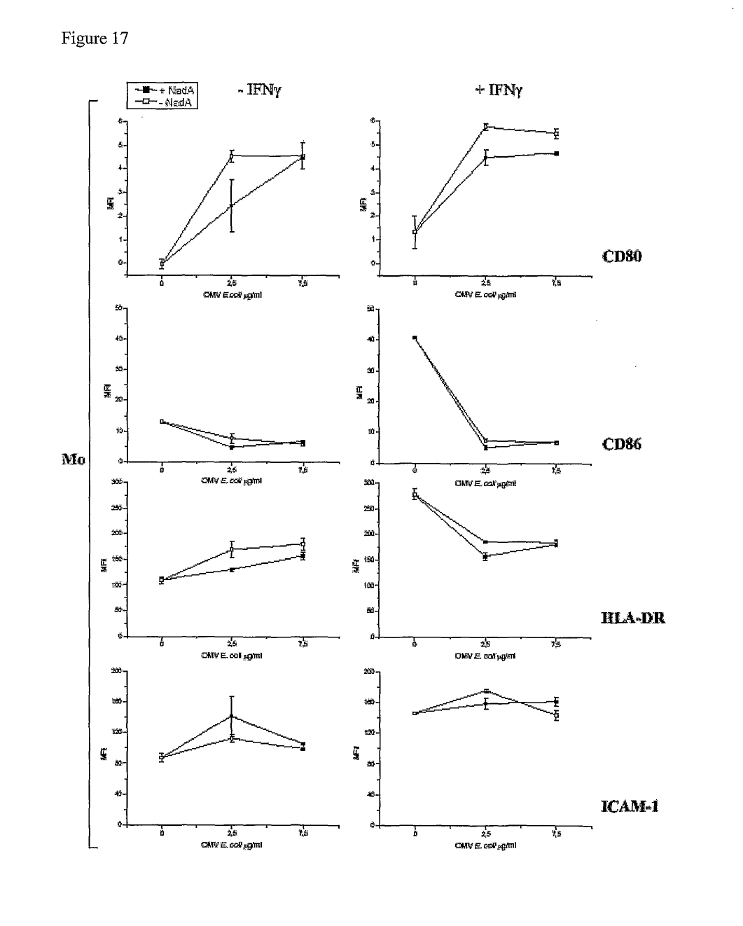

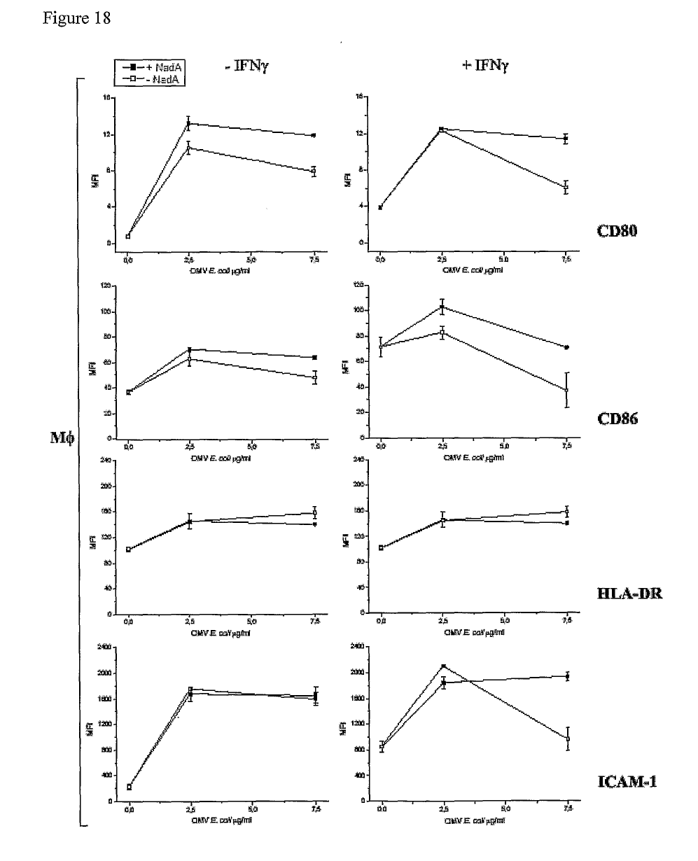

In Neisseria OMV-treated monocytes, no significant difference on marker expression was seen between OMV_{wt} and OMVₖₒ (FIG. 19). CD80 expression on macrophages was not significantly increased after exposure to OMV_{wt} with IFN-γ co-stimulation. In macrophages treated with both OMV_{wt} and OMVₖₒ no significant difference in expression of CD86, HLA-DR, and ICAM-1 was seen (FIG. 20).

The results suggest that in both monocytes and macrophages, recombinant soluble NadA increases the antigen presenting activity by up-regulating the expression of co-stimulatory molecule CD80 (INFγ-dependent) and the adhesion molecule ICAM-1 (INFγ-independent). When the cells were treated with E. coli OMV or Neisseria OMV, no significant difference was observed due to the presence of other immuno-modulatory components on the bacterial membrane surface.

### Effect of NadA_{Δ351-405} on Cytokine and Chemokine Secretion by Human Monocytes and Macrophages

Since cytokine and chemokine secretion was noted during cell activation, experiments were carried out to determine whether soluble NadA or the protein expressed on the surface of E. coli or N. meningitis OMV was responsible for this.

The secretion of various immune mediators by isolated adherent human lymphocytes and macrophages was assayed with a Bio-Plex immune array. The pro-inflammatory cytokines IL-1α, IL-1β, TNFα, IFNγ, IL-6, the growth factor GM-CSF, the regulatory cytokines IL-12 (p40), IL-12 (p70), IL-10, as well as the chemokines IL-8, MCP-1, MIP-1α, IP-10, RANTES and EOTAXIN were assayed. The lymphocyte cytokines IL-2, IL-3, IL-4, IL-5, IL-7, IL-13 and IL-15 were also assayed. IL-23 expression was assayed using an ELISA assay. No secretion of IL-2, IL-3, IL-4, IL-5, IL-7, IL-13 or IL-15 was detected.

Peripheral monocytes and macrophages were stimulated with different concentrations of NadA_{Δ351-405}, with or without LPS (0.2 μg/ml) and IFNγ (1000 U/ml), and with purified E. coli or N. meningitis OMV.

### NadA, LPS and IFNγ Effect on Cytokine and Chemokine Secretion

The effect of soluble NadA_{Δ351-405} with co-stimulation by IFNγ and/or bacterial stimulus LPS was tested. NadA_{Δ351-405} was found to induce the secretion of the cytokines IL-1α, IL-1β, TNFα, IFNγ, IL-6, GM-CSF, IL-12 (p40), IL-12 (p70), IL-10, and the chemokines IL-8, MCP-1, MIP-1α, LP-10, RANTES and EOTAXIN.

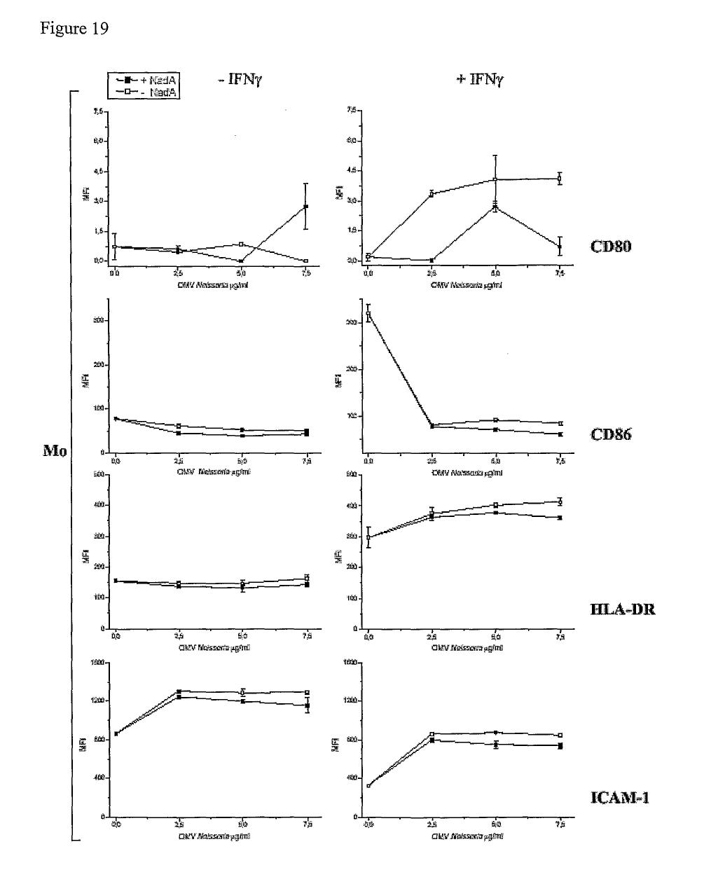

IL-1α, IL-1β and TNFα (FIG. 21) were not significantly induced by NadA, but the presence of IFNγ induced expression of TNFα. Moreover, upon co-stimulation with LPS, the expression of IL-1α, and particularly TNFα, were inhibited by NadA_{Δ351-405}. Conversely, IL-10 expression was efficiently increased by NadA, but only in the presence of IFNγ and LPS.

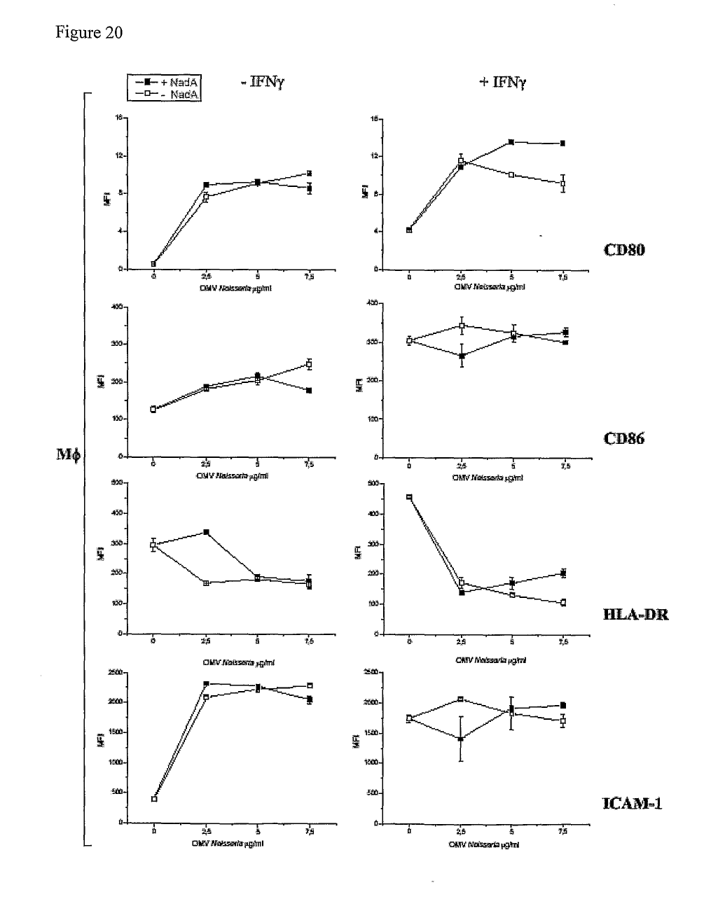

Macrophages incubated with NadA produced only IL-1β and TNFα, but much less than produced by monocytes. No IL-1α was produced (FIG. 22). In the presence of IFNγ, IL-1β levels decreased, but TNFα levels increased. When the cells were incubated with NadA and LPS there was an inhibitory effect, compared to monocytes.

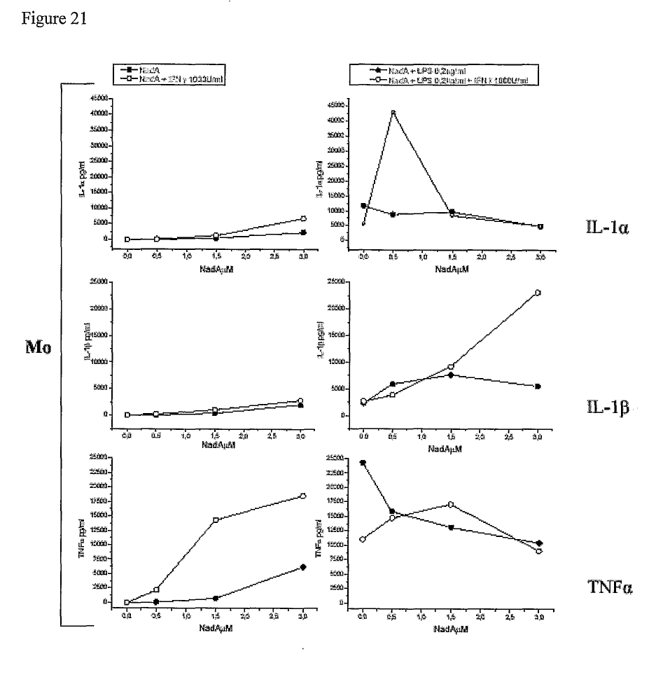

NadA_{Δ351-405} was able to induce significant secretion of IL-6 by monocytes, both in the presence or absence of LPS. This was increased upon co-stimulation with IFNγ (FIG. 23). However, NadA_{Δ351-405} does not stimulate secretion of GM-CSF, even with IFNγ co-stimulation, but LPS does appear to have an effect.

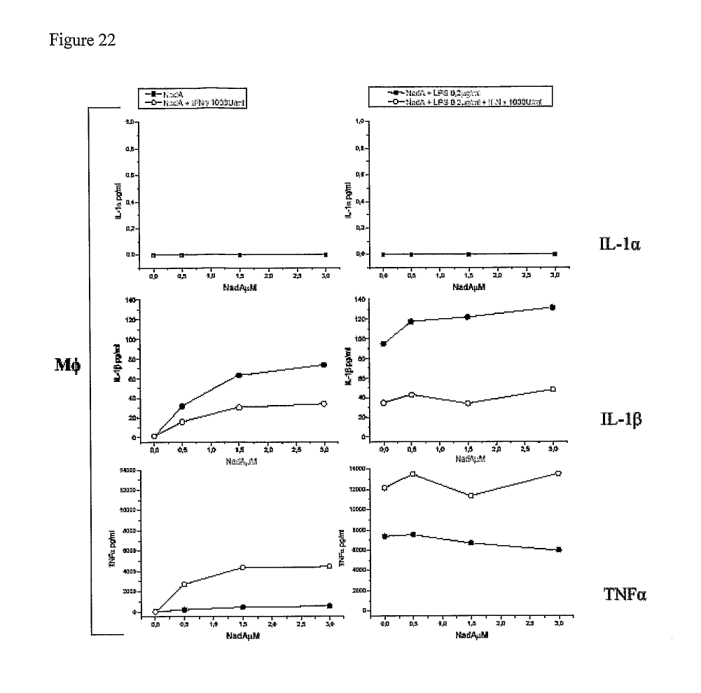

NadA_{Δ351-405} together with IFNγ produced increasing secretions of IL-6 by macrophages (FIG. 24); but when incubated with LPS, IL-6 levels decreased. No secretion of GM-CSF was detected.

Therefore NadA_{Δ351-405} induced IL-6, mainly in monocytes, which could induce macrophage maturation. This hypothesis is supported by the inhibitory effect seen on GM-CSF expression.

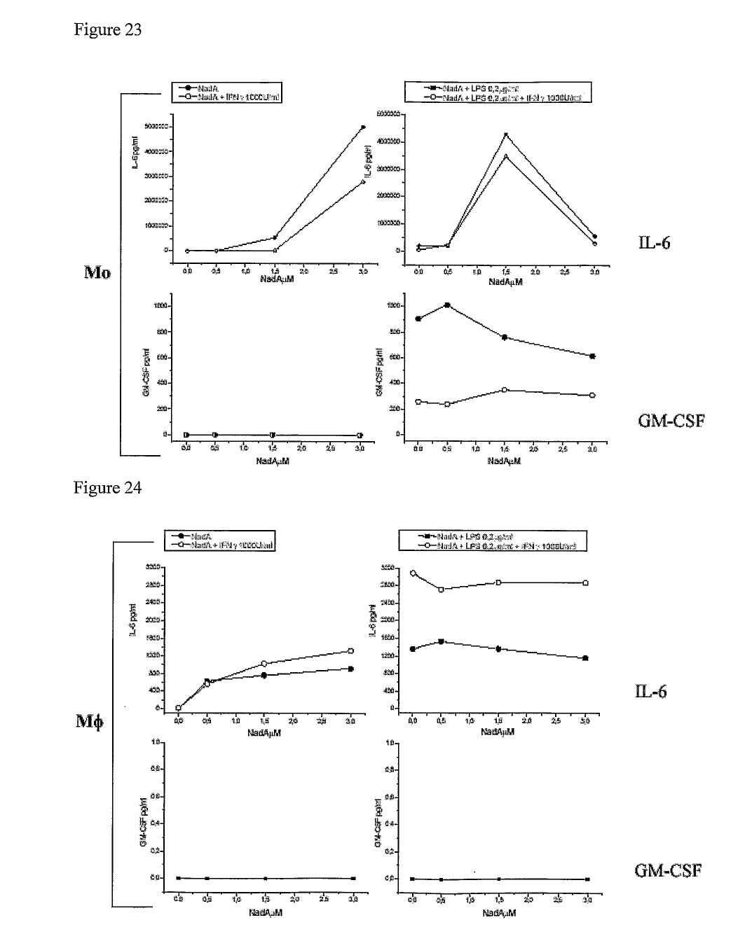

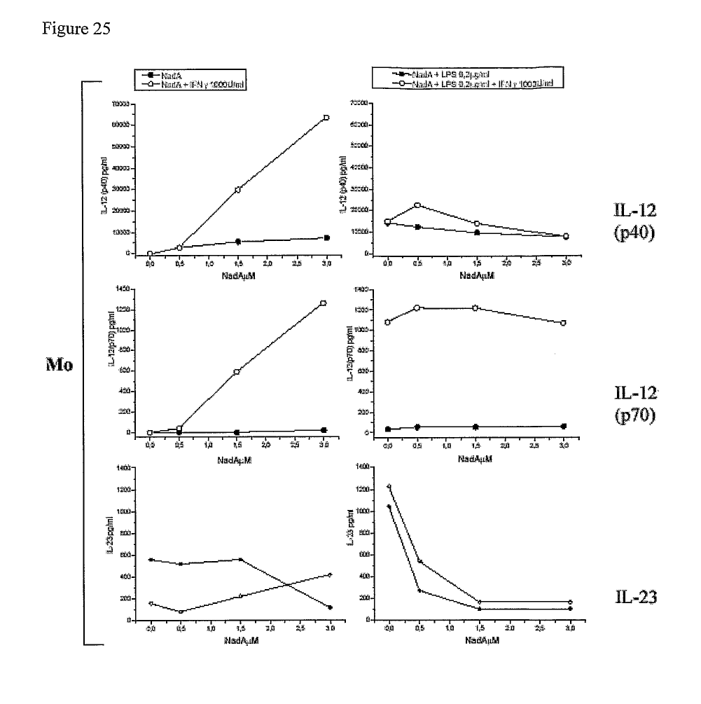

IL-12(p40) and IL-12(p70) were not significantly expressed in monocytes stimulated by NadA_{Δ351-405}, alone or in the presence of LPS (FIG. 25), but expression was noted upon co-stimulation with IFNγ. NadA_{Δ351-405} induced IL-23 expression only in the presence of IFNγ, co-stimulation with LPS or NadA_{Δ351-405} stimulation alone resulted in a decrease of IL-23 expression. The effect was similar in macrophages, but stimulation with NadA_{Δ351-405} alone resulted in a decrease of IL-12(p40) expression even in the presence of LPS and IFNγ (FIG. 26).

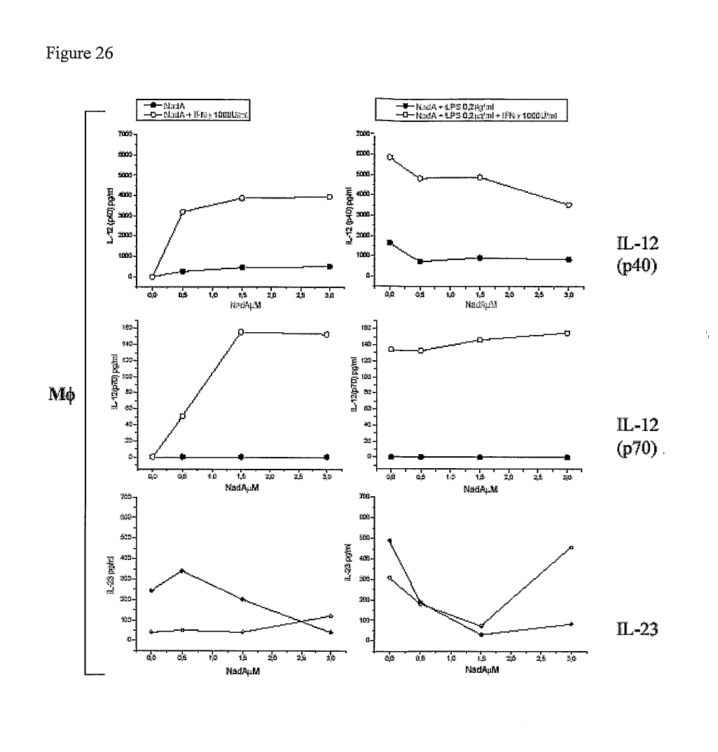

Macrophages produced more IL-10 than monocytes (FIG. 27). In both cases, NadA induced the production while LPS modulated the effect; IFNγ induced a decrease of IL-10 expression independent of NadA stimulation. This effect on IL-10 could result in the induction of a Th2 response.

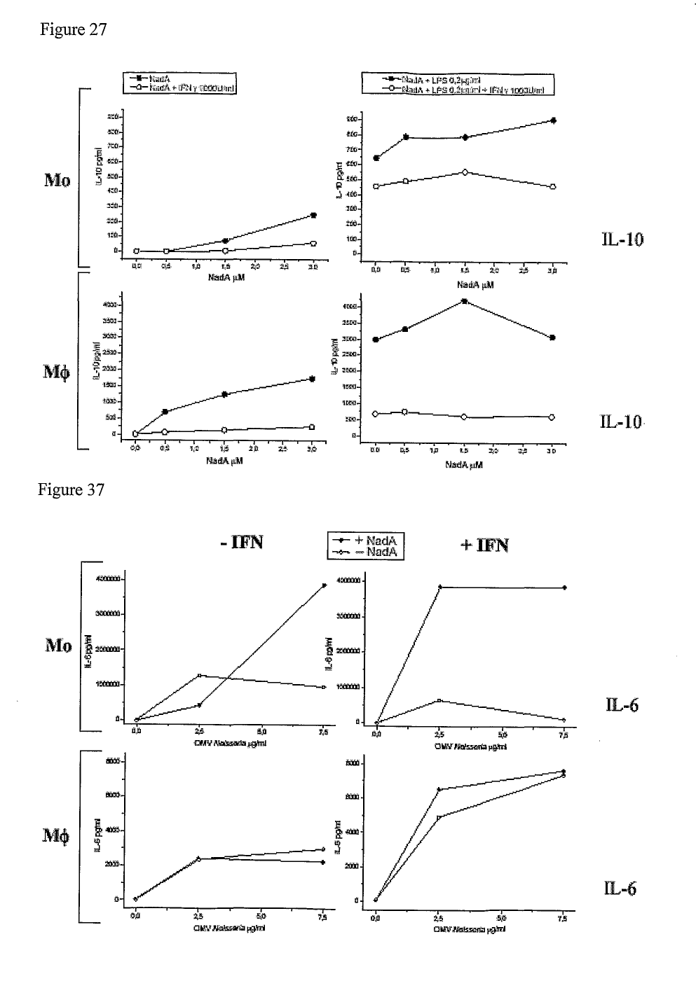

NadA_{Δ351-405} alone induced a significant secretion of IL-8, whereas co-incubation with IFNγ resulted in a decrease of secretion levels (FIG. 28). A similar, though less extreme secretion was seen with LPS. Monocytes also produced significant levels of MCP-1 upon NadA stimulation, and in the presence of LPS the effect was increased, but was decreased with IFNγ. RANTES expression was increased by any of the stimuli. MIP-1α was produced upon NadA stimulation, with or without co-stimulus; LPS had no significant effect on this.

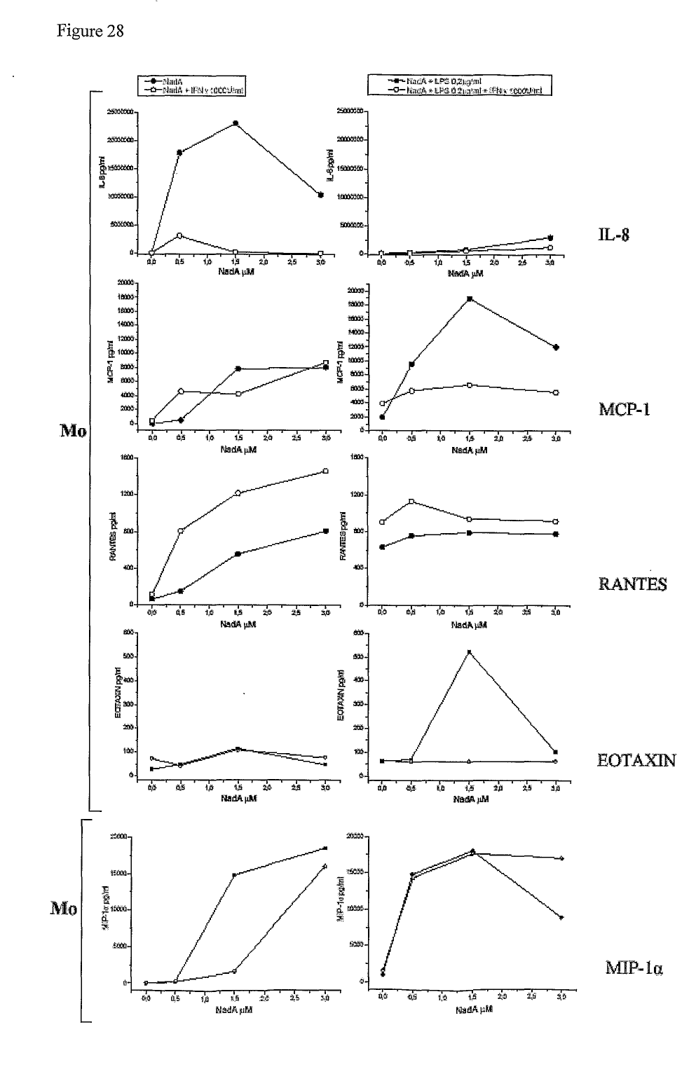

IL-8 and MCP-1 were also expressed in macrophages, but the expression was lower compared to that seen in monocytes (FIG. 29). The secretion of RANTES was also similar, but LPS resulted in a decrease of expression. Moreover, NadA induced a decrease in EOTAXIN expression but in the presence of LPS expression was increased, but IFNγ had no effect. MIP-1a production in macrophages was similar to that seen in monocytes.

In neither monocytes nor macrophages was NadA alone able to produce IP-10, but upon co-stimulation with IFNγ, secretion was observed but was negatively modulated in macrophages. Both of these cell models vary in levels of IP-10 secretion when incubated with NadA, with or without LPS or IFNγ. Secretion of IFNγ was the same in monocytes or macrophages—NadA, with or without LPS, was not able to stimulate production. However, when incubated with an immunological co-stimulus, there was an increase in IFNγ production but this did not appear to be dependent on NadA stimulation, except in macrophages, but only in the absence of LPS.

NadA alone induced the secretion of IL-8, MIP-1α and RANTES, in both monocytes and macrophages, and in the presence of LPS, MCP-1 expression was also seen.

Monocyte Stimulation with OMV from E. coli

In order to evaluate functional properties of the immune system cells under conditions similar to physiological conditions, monocytes and macrophages were stimulated with outer membrane vesicle preparations, obtained from a strain of E. coli (E. Coli pETBL21), and alternatively with OMV expressing NadA (OMV_{NadA} or OMV_{pET}).

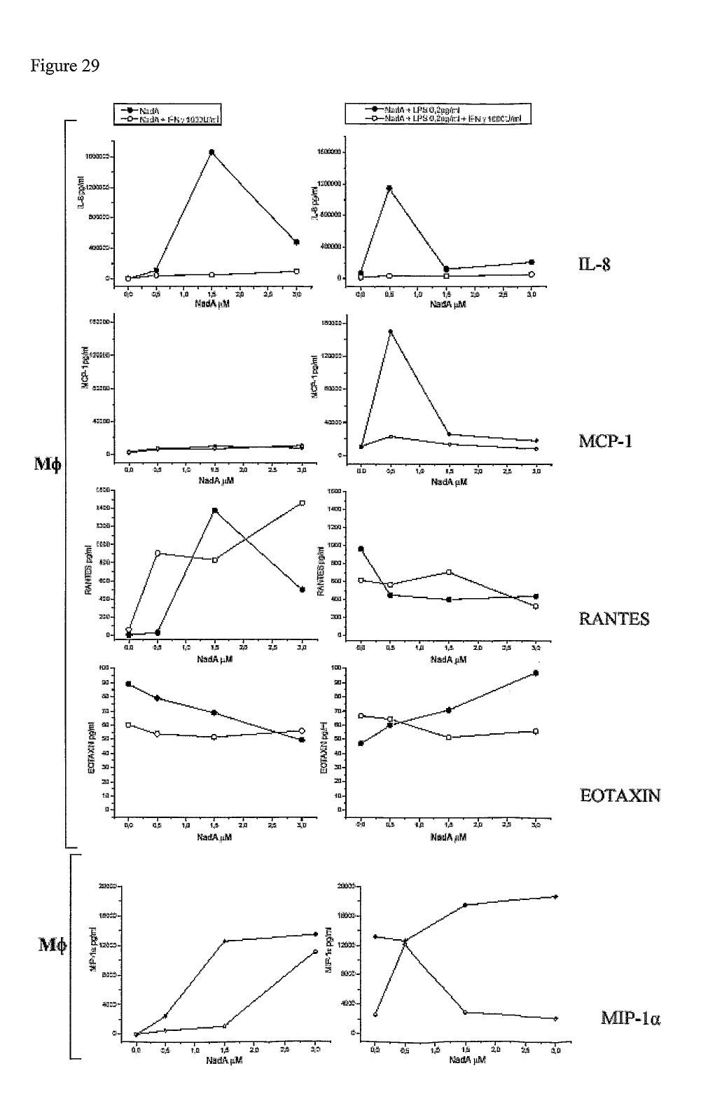

In normal conditions, monocytes secrete IL-1α, TNFα and IL-1β, while macrophages only secrete IL-1β and TNFα (FIG. 30). In monocytes, cytokine expression was only induced upon OMV_{NadA} treatment when cells were also treated with IFNγ. In macrophages, IFNγ induced a reduction of IL-1β production in OMV_{NadA}-treated cells, whereas TNFα secretion was induced by OMV_{NadA} only in the absence of IFNγ.

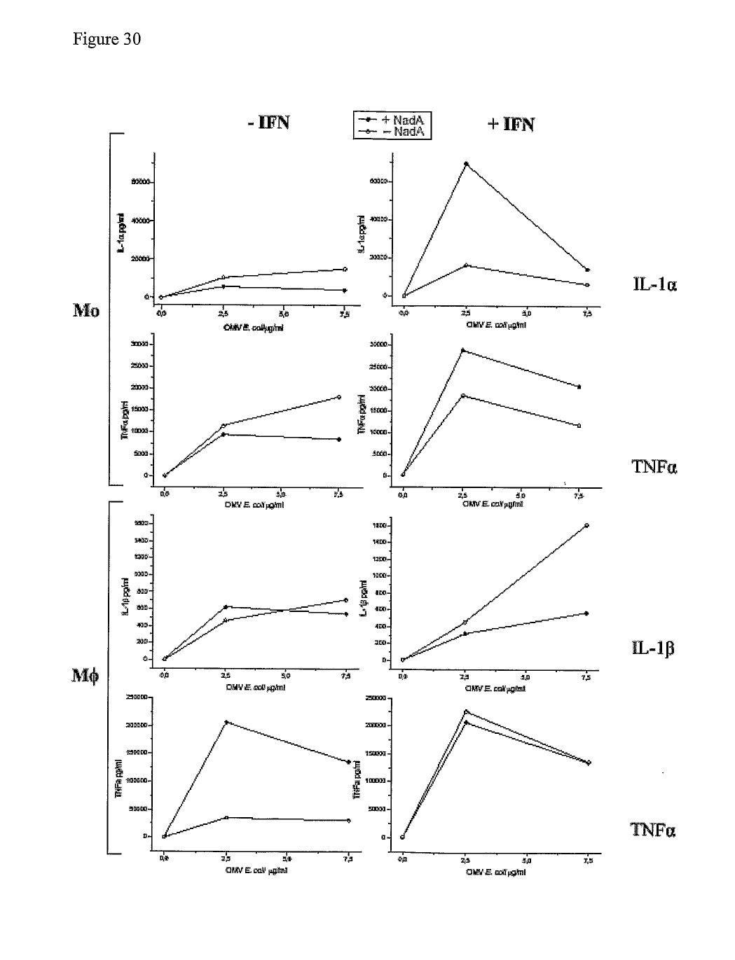

IL-6 secretion (FIG. 31) was inhibited upon OMV_{NadA} treatment, but was completely abolished in IFNγ-treated cells. In macrophages, IL-6 was released only when cells received an immunological co-stimulus and when they were treated with OMV_{pET}.

Only monocytes produced GM-CSF, and the secretion was up-regulated by OMV_{NadA} only in IFNγ-treated cells.

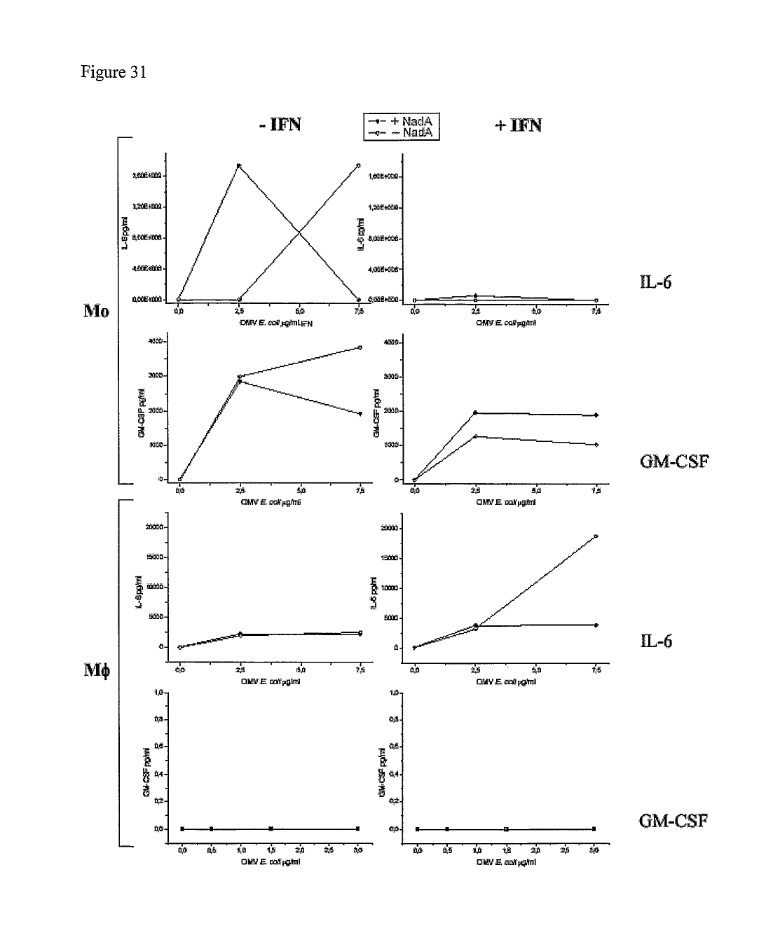

The secretion of the regulatory cytokines IL-12 (p40) and IL-12 (p70) (FIG. 32) was induced at the same levels upon OMV treatment. IL-10 was secreted from monocytes only when cells were stimulated with OMV_{NadA} plus IFNγ. IL-23 secretion was induced only in cells treated with OMV_{pET}.

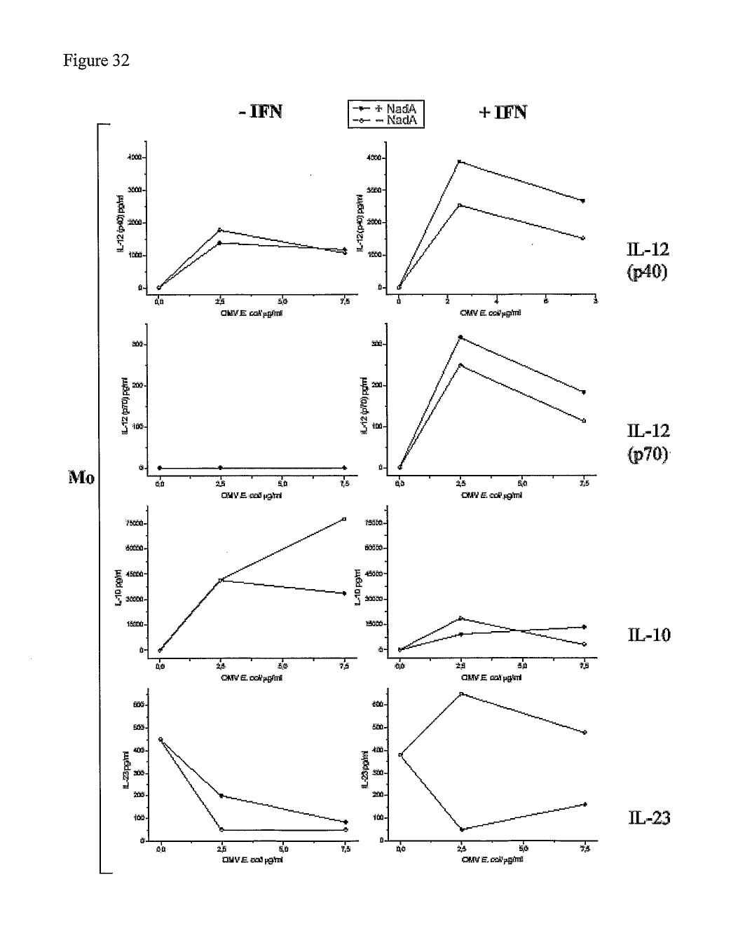

In macrophages (FIG. 33), IL-12(p40) and IL-12(p70) secretion was similar in cells stimulated with OMV, but IFNγ induced an up-regulation of secretion, in particular in OMV_{pET}-treated macrophages. In these cells, IL-23 was significantly released only after IFNγ treatment; together with the immunological co-stimulus, OMV_{NadA} induced a greater secretion of IL-23. The level of IL-10 secretion was similar.

OMV from both strains of E. coli were able to stimulate cells to produce regulatory cytokines, even though monocyte and macrophage responses were opposite.

The chemokines IL-8, MCP-1, IP-10, RANTES and MIP-1α were secreted from both monocytes and macrophages.

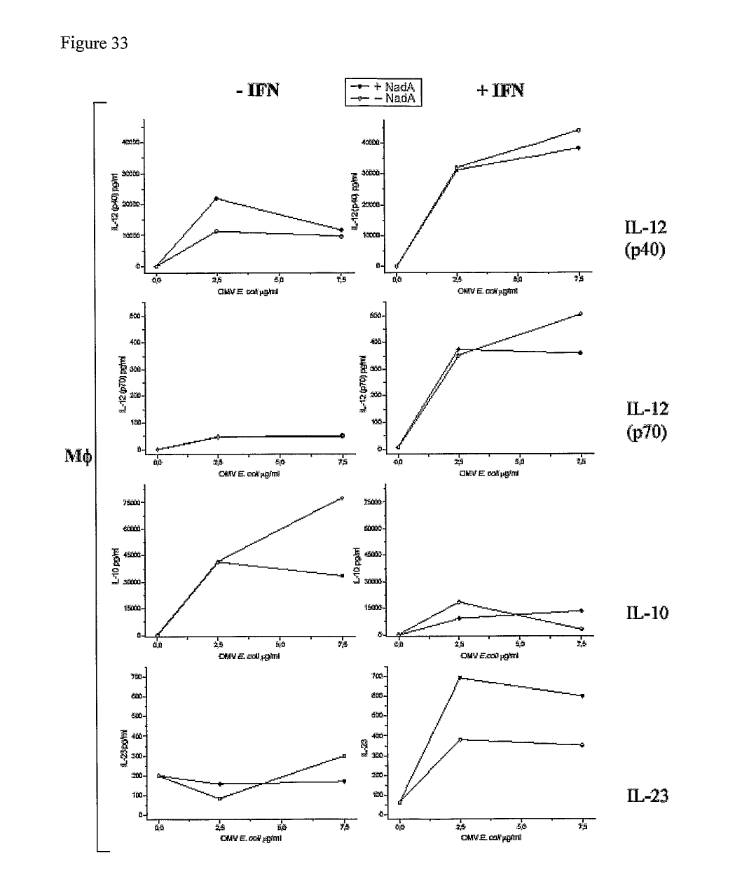

IL-8 (FIG. 34) was equally produced by both OMV_{NadA}- and OMV_{pET}-treated monocytes; in the presence of IFNγ, cells stimulated with OMV_{NadA} produced a little more chemokine compared to the control. The same results were obtained for MCP-1 and IP-10. RANTES secretion was induced in cells treated with both stimuli, with or without IFNγ.

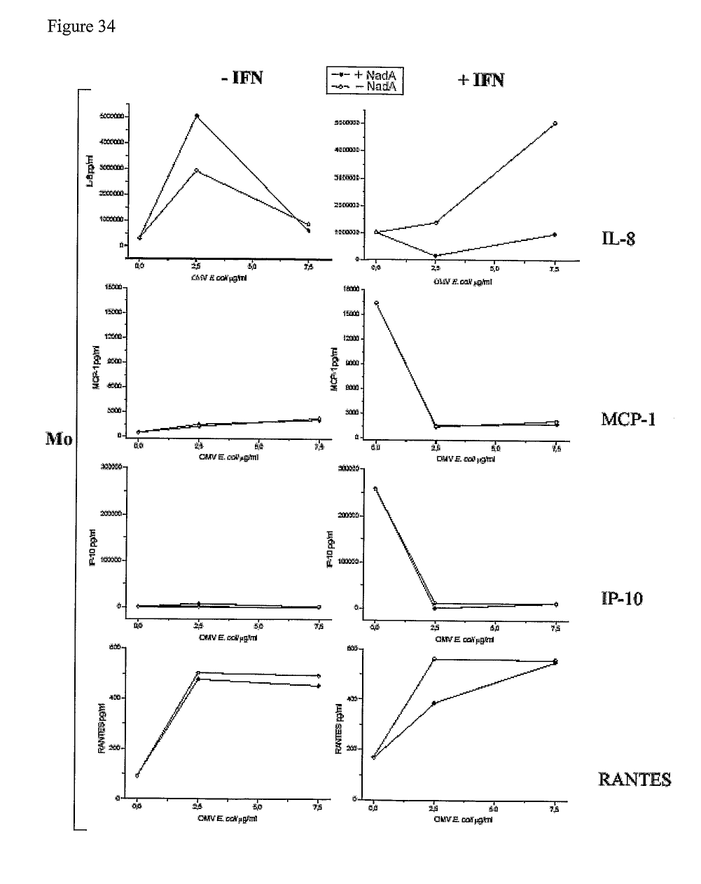

In macrophages (FIG. 35), IL-8 and IP-10 were produced at the same levels as in monocytes. However, MCP-1 was produced upon OMV_{NadA} stimulation, but this secretion was inhibited in the presence of IFNγ, by the same amount for both types of OMV.

Stimulation with OMV from Neisseria meningitidis MC58

In order to mimic the in vivo stimulation, cells were treated with OMV obtained from the Neisseria meningitidis strain MC58 (OMV_{wt}) or the mutant strain lacking NadA expression (OMVₖₒ).

In monocytes, the production of IL-1α, IL-1β, IL-6, IL-12 (p40), IL-12 (p70), IL-10, IL-8, IP-10, MCP-1, RANTES and also TNFα and MIP-1α was observed (but the latter were overproduced). Macrophages secrete IL-10, TNF-α, IL-6, IL-12 (p40), IL-12 (p70), IL-10, IL-8, IP-10, MCP-1, MIP-1α and RANTES, but RANTES was produced in excess and therefore not measurable.

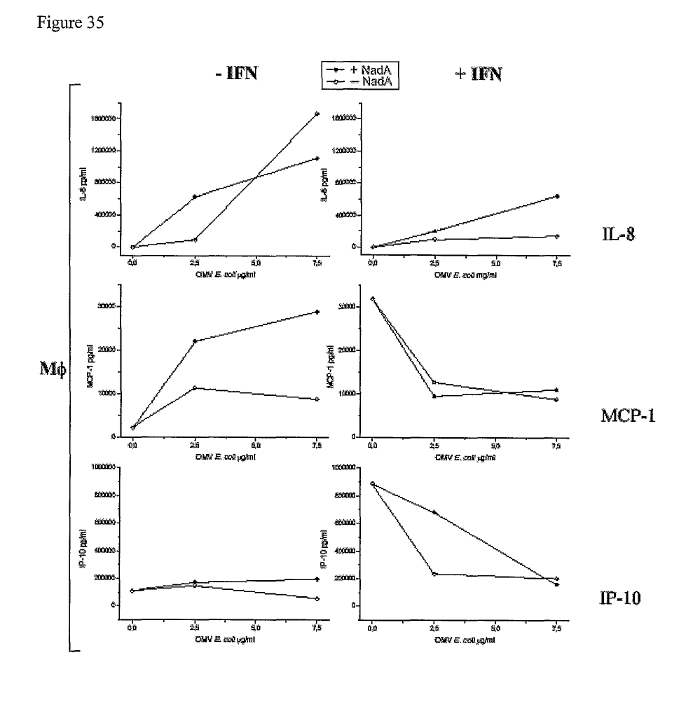

In monocytes (FIG. 36), OMV_{wt} stimulated IL-1α secretion more than OMVₖₒ, but only in the presence of IFNγ. The immunological co-stimulus favours the induction of TNFα secretion especially in cells treated with OMV expressing NadA.

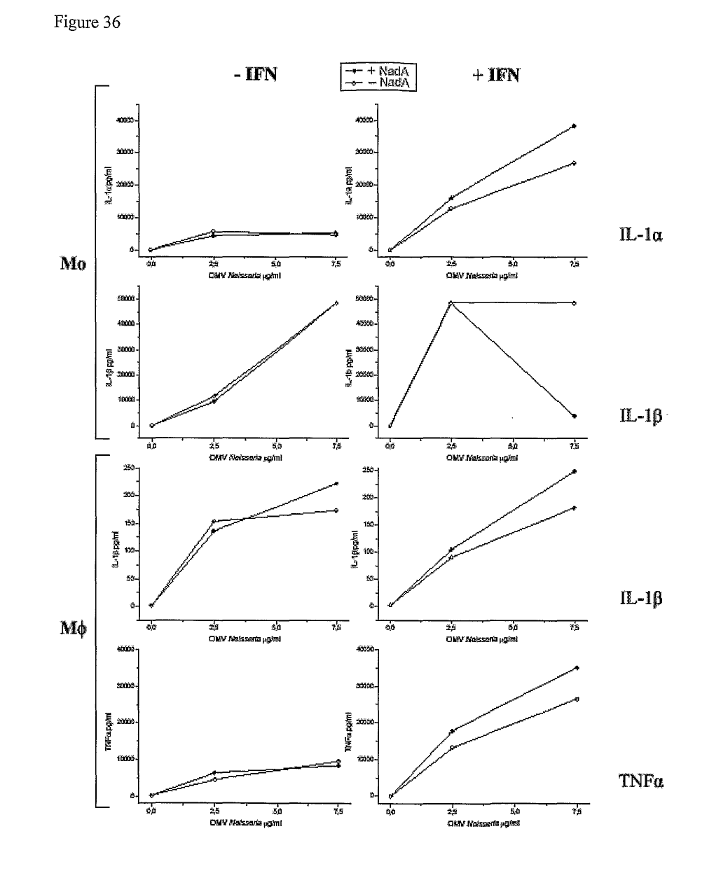

IL-6 secretion (FIG. 37) is more induced in monocytes stimulated with OMV_{wt} in the presence or absence of IFNγ; in macrophages the response is similar for both OMV types.

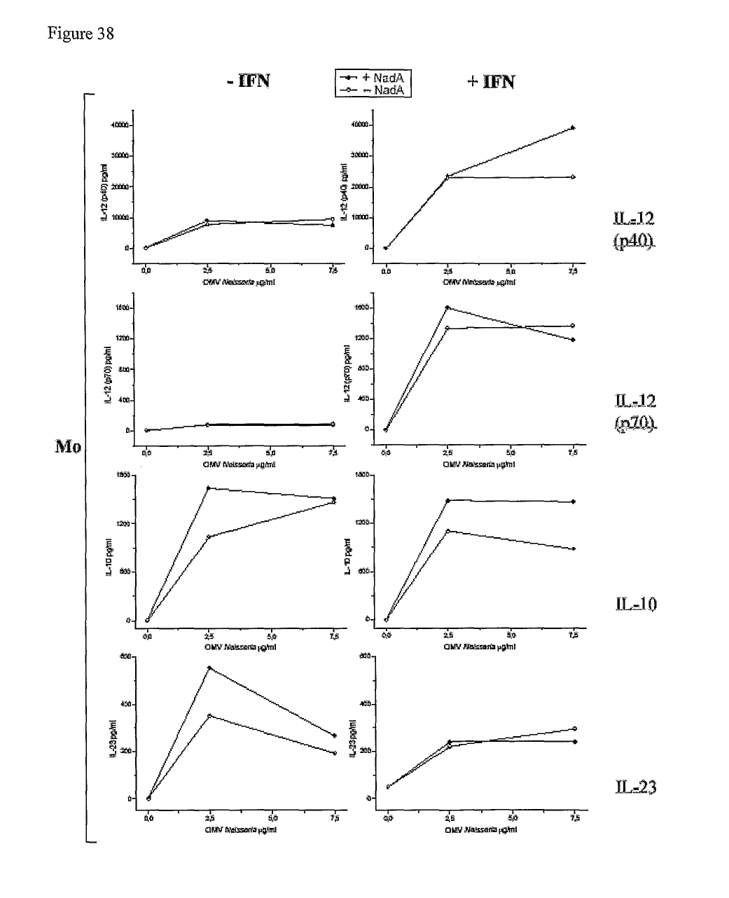

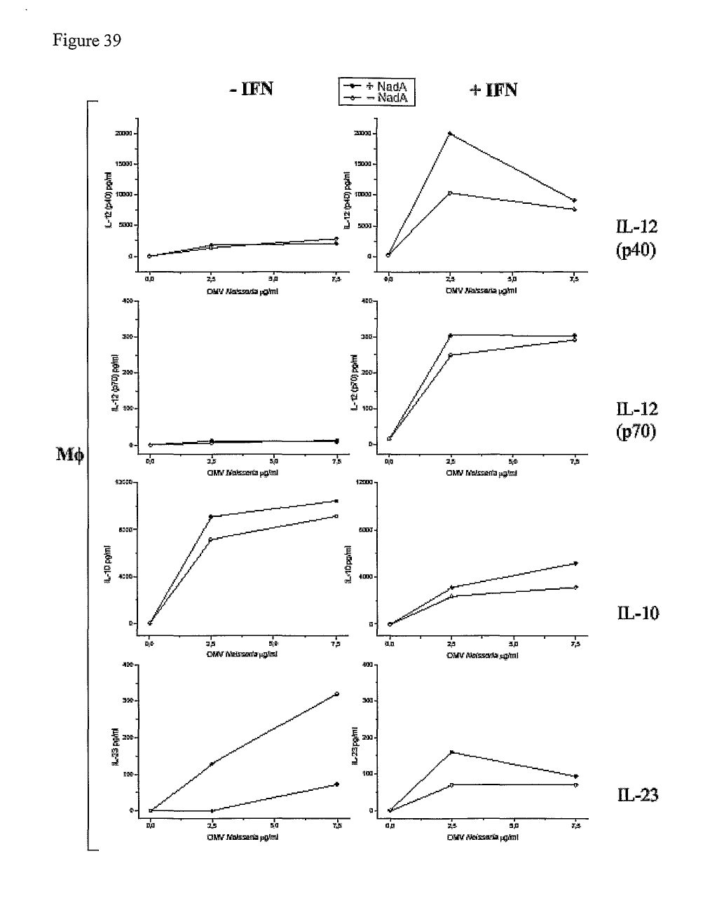

The secretion of the regulatory cytokines (FIGS. 38 and 39) IL-12 (p40) and IL-12 (p70) is induced by both OMV_{wt} and OMVₖₒ, at the same levels in monocytes and macrophages and only in the presence of IFNγ. Moreover, IL-12 (p40) production is notably higher in comparison with IL-12 (p70). In monocytes, IL-10 is induced mainly by OMV_{wt} in the presence of IFNγ. In macrophages, OMV expressing NadA stimulate cytokine secretion more than OMVₖₒ, with or without IFNγ. IL-23 production in monocytes is induced mainly by OMV_{wt} compared with OMVₖₒ, unlike in macrophages. In neither monocytes nor macrophages are any significant differences observed in the presence of IFNγ.

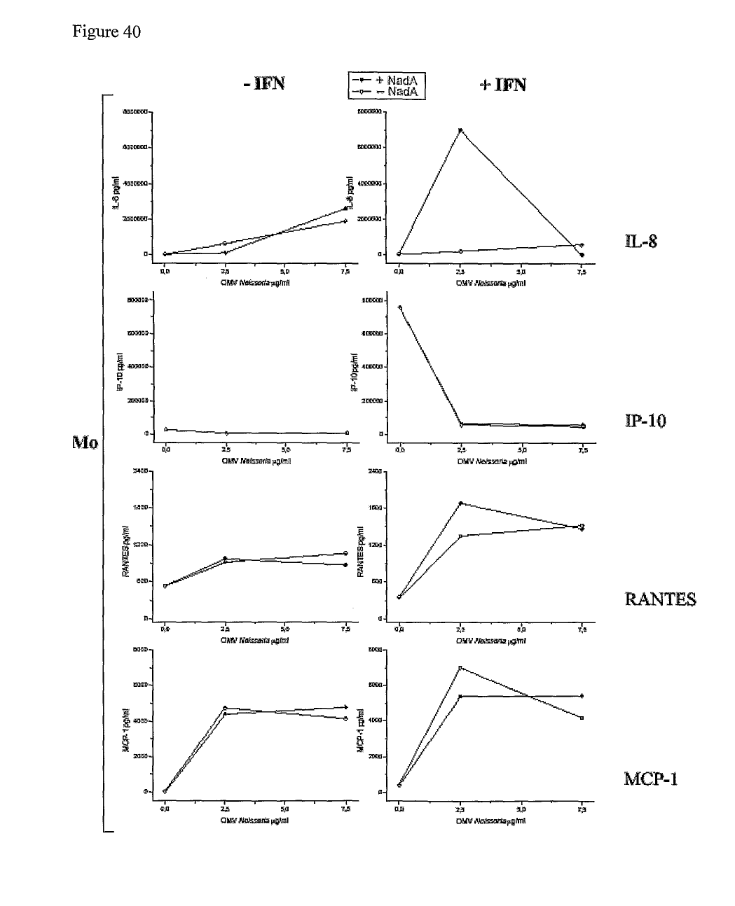

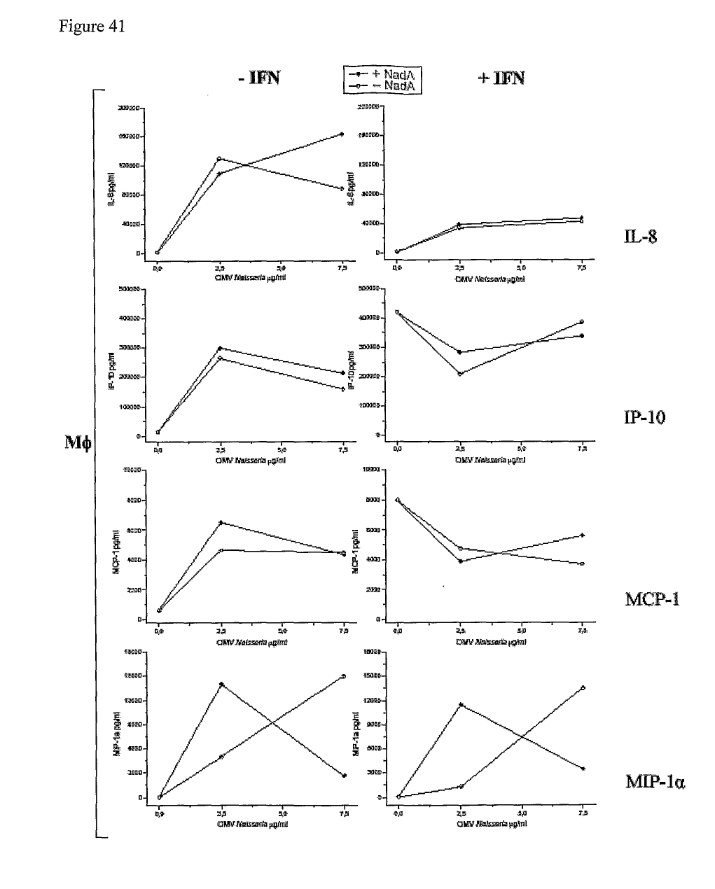

In monocytes (FIG. 40), IL-8 secretion is extremely elevated, in comparison with that induced in macrophages (FIG. 41). In both types of cells, OMV_{wt} and OMV_{NadA} induce the same amount of chemokine in the presence of IFNγ. In the absence of IFNγ, vesicles expressing NadA induce greater secretion, measurable only in macrophages.

IP-10 is not secreted in monocytes stimulated with either type of OMV, but in the presence of IFNγ a high production was observed in control cells, which was inhibited irrespective of whether OMV_{wt} or OMVₖₒ was used. In macrophages, IP-10 secretion was stimulated in similar amounts when the cells were incubated with either OMV preparation, but in the presence of IFNγ a decrease of IP-10 was observed compared with control cells. RANTES secretion in monocytes was induced upon immunological co-stimulation, but the amounts were similar for both types of OMV.

In monocytes, vesicles stimulated MCP-1 secretion both in the presence and absence of IFNγ. In macrophages an increase of MCP-1 production upon OMV_{NadA} stimulation was observed, but the presence of IFNγ had an inhibitory effect. Moreover, in macrophages, MIP-1α was produced at higher levels in OMVₖₒ-stimulated cells, compared with OMV_{wt}-stimulated cells, with or without IFNγ.

In conclusion, NadA is able to induce the secretion of cytokines and chemokines, both in monocytes and in macrophages. It is interesting to note that in both types of cells, the production of pro-inflammatory and vasoactive cytokines, like IL-1α, IL-1β and TNFα, is induced only at low levels in the absence of IFNγ. NadA has a great effect on chemokine production, especially on IL-8, and it is able to modulate IL-6 and IL-10 secretion.

These data indicate that the protein is a good adjuvant as a vaccine should induce the expression of the co-stimulatory molecules necessary for the activation and differentiation of T lymphocytes, without exacerbating inflammation.

Survival, Differentiation and Stimulation of Human Monocyte Incubated with NadA

Peripheral blood monocytes can differentiate into dendritic cells or macrophages depending on the environmental factors encountered during their migration from the blood to peripheral tissues. Monocytes have a limited life span, and their homeostasis is regulated by programmed cell death in vivo. The onset of apoptosis can be prevented by activating factors such as both microbial or endogenous stimuli. These monocytes have a prolonged survival and they can differentiate into other cell types and contribute to the establishment of immune responses by the secretion of soluble mediators.

### Survival Analysis of Human Monocyte

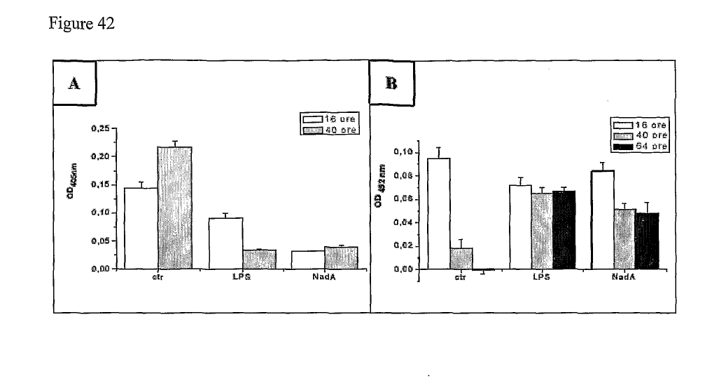

The survival effect of NadA on monocytes was investigated. The meningococcal protein induced an apoptotic effect that was four times less than in medium-treated monocytes and two times less than the amount induced by LPS. Apoptosis was not increased after a 40 hour exposure to NadA_{Δ351-405} or LPS. However, the amount induced by stimuli was showed to be very much alike. NadA survival effect was compared with the action of LPS or medium alone. The data showed a similar induction by protein and endoxin, in contrast with monocytes treated with medium that quickly died (FIG. 42). These data suggest that the meningococcal protein induced anti-apoptotic intracellular signalling in monocytes.

### Morphological Analysis of Human Monocyte

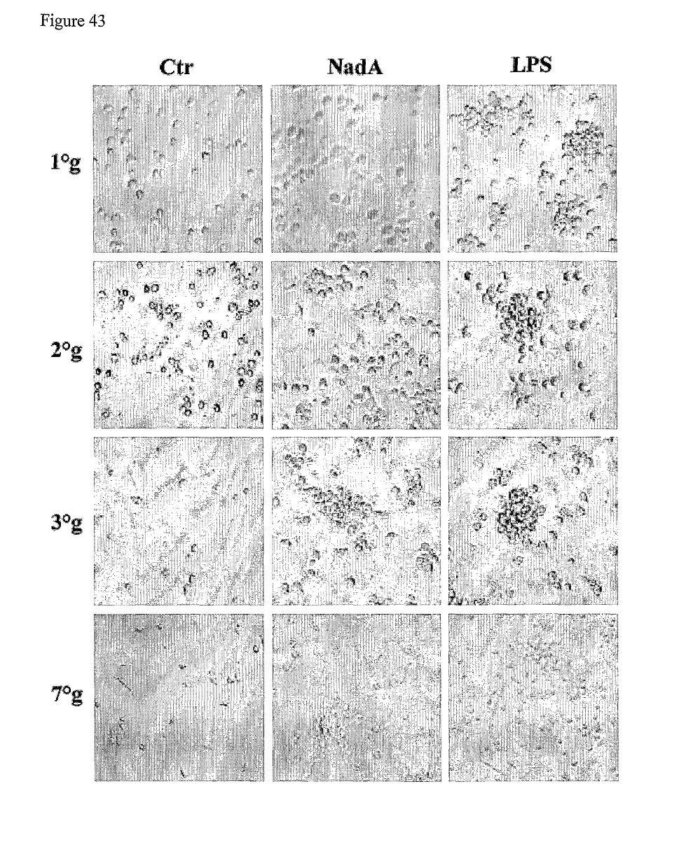

In order to evaluate the possible long-term, differentiation effect of NadA, adherent monocytes were treated with medium alone, NadA_{Δ351-405} or E. coli LPS and cells were cultured for seven days. At day 4 the agonists were added again to ensure a constant stimulation. Cell morphology was monitored by light microscopy after 1, 2, 3 and 7 days. Apoptosis in monocytes treated with medium alone was noted after 3 days of culture, and surviving cells displayed a macrophage-like morphological heterogeneity. In contrast, monocytes after a 3-day incubation with the meningococcal protein became elongated and tended to cluster but the clustering effect was not as strong as that seen following stimulation with LPS. Both elongated cell morphology and cell-widespread distribution in the well was seen in monocytes treated with NadA after 7 culture days. Furthermore, the number of surviving cells was greatly increased with respect to the control. The cells treated with NadA or LPS displayed a macrophage-like morphological heterogeneity (FIG. 43). NadA_{Δ351-405} thus induces both alteration cell morphology and distribution, this is a good indicator of monocyte activation and differentiation.

### Analysis of Human Monocytes Surface Markers

The long-term, differentiation effect of NadA_{Δ351-405} on monocytes was further investigated by measuring the expression of the antigen presentation marker MHC-II, co-stimulatory molecules CD80 and CD86 and cell-specific molecules: CD14 (monocyte), CD16 (macrophage) and CD1a (Dendritic cell).

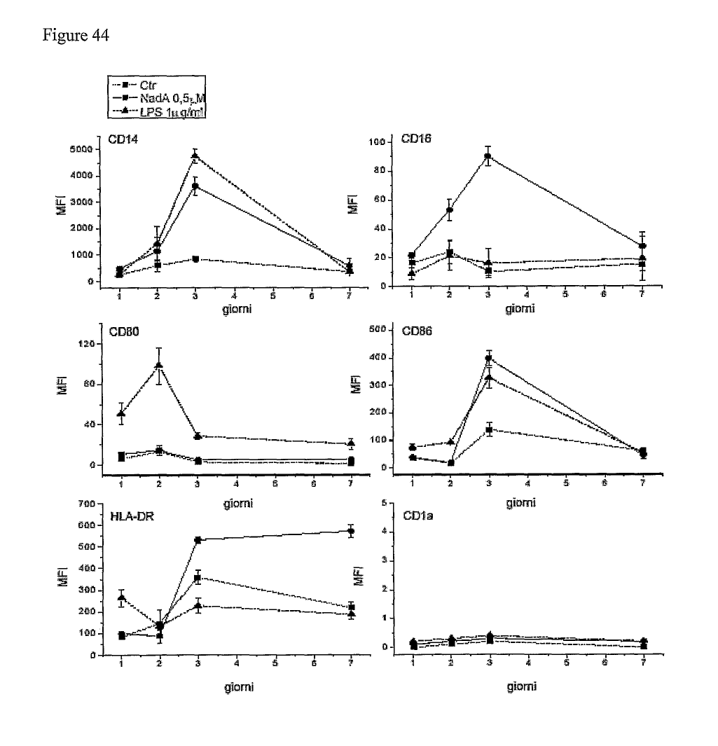

CD14 expression by monocytes treated with NadA steadily increased on days two and three, but then decreased so that on day 7 its level was not significantly different with respect to control cells. The NadA effect was thus very similar to that of LPS (FIG. 44).

CD16 expression on NadA-treated cells also increased more intensely than in the control cells in the first three days, but decreased thereafter. In this case, however, stimulated cells showed a CD16 level significantly higher than control cells. CD16 on LPS-treated monocytes did not show such a peak of expression in the first few days, but showed lower expression levels compared to control cells. After seven days, however, CD16 expression was as in the control cells.

CD80 was not over-expressed with respect to the control cells, while CD86 expression on NadA-treated cells increased more intensely than in the control cells in the first three days, and decreased thereafter. LPS induced a transient peak of expression on day two (CD80) or three (CD86). After seven days incubation with LPS, CD80 expression was higher than in control cells, while CD86 expression was not significantly different.

HLA-DR surface expression was slightly increased by LPS after one day, but then decreased to reach a final value after seven days very similar to controls. In comparison, HLA-DR levels on monocytes treated with meningococcal adhesin was as in control cells after two days, and reached a maximal expression level on day three, which remained constant until day seven.

Dendritic marker CD1a expression was not increased by either NadA or LPS.

The results showed that prolonged incubation with NadA_{Δ351-405} supports monocyte survival in vitro and differentiation into a CD14⁺, CD16⁺, HLA-DR⁺, CD80⁻, CD 86⁻, CD1a⁻, macrophage-like phenotype after 7 culture days. Measurement of surface markers suggest that NadA_{Δ351-405} induces a cell phenotype competent for antigen presentation, only after 3 days of culture, but not after 7 days. Upon 7 days NadA stimulation, it was noticed that the bacterial adhesin, compared to LPS, was not over activating antigen presenting activity, since the expression of co-stimulatory molecules CD-80 and CD-86, necessary for efficient T lymphocytes activation was not increased. However, the upregulation of CD14, the co-receptor of LPS, on the 3rd day suggests that NadA, like LPS, improves the innate binding capability of bacterial microbes and of their products and promotes cell survival. NadA, like LPS, increases the expression of FcRgIII-CD16, and therefore seems to improve the binding capacity of microbes and microbial products mediated by antibodies.

### Analysis of Soluble Mediator Secretion

The secretion of the main immune mediators by human monocytes, after 3 and 7 culture days following stimulation with NadA_{Δ351-405} or LPS, was tested with a Bioplex immune array after 24 hours incubation with LPS. Analysis was performed to test pro-inflammatory cytokine TNFα and IL6, regulatory cytokine IL-10 and IL-12(p70) and chemokine IL-8, MCP-1, MIP1-α, RANTES and LP-10 secretion.

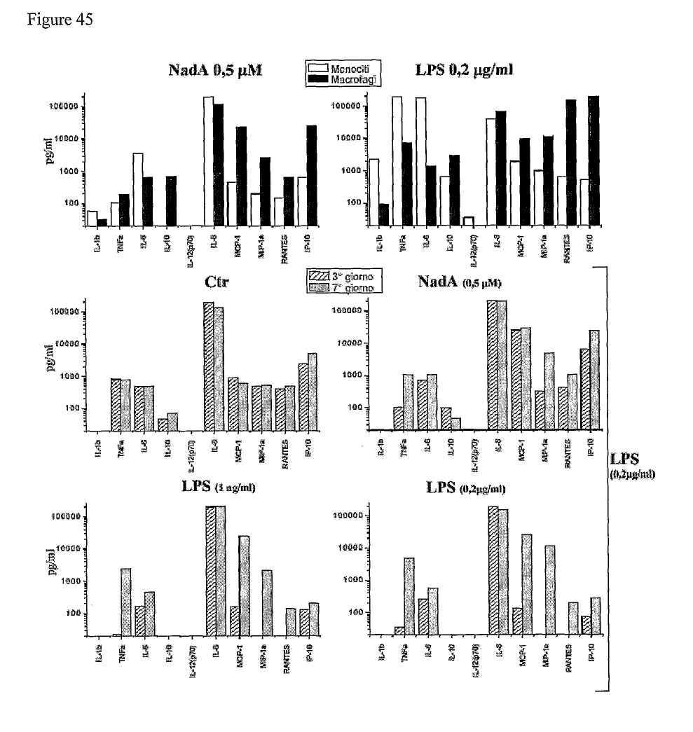

At the 3rd and 7th culture day no secretion of IL-10 and IL-12(p70) was detected (FIG. 45). The secretion pattern was similar to that of the control cells treated with medium alone after both 3 and 7 days. Monocytes cultured with NadA_{Δ351-405} and then stimulated with LPS showed secretion of the tested mediators. At the 3rd culture day, chemokine production was greater than after 7 days of cell culture. NadA_{Δ351-405} induced a greater production of IL-6 compared to LPS-treated cells, which was clearly visible after 3 days of culture. Monocytes treated with NadA_{Δ351-405} were able to induce IL-10 production, in contrast to that seen for LPS-treated cells, which were not able to induce IL-10 secretion under any condition. LPS-cultured cells were shown to be less responsive than NadA-cultured monocytes after re-stimulation by LPS. Cytokine and chemokine secretion patterns were closely associated with the macrophage phenotype secretion pattern, which showed a strong pro-chemokine effect.

The results show that NadA induces anti-apoptotic intracellular signaling and cellular survival. This meningococcal protein induces a macrophage-like phenotype capable of efficient innate and adaptive capture, without increasing lymphocyte activation and hence the amplification of inflammatory reactions. In addition, NadA has been shown to be biologically active on monocytes, inducing a profile of extracellular signals favouring monocyte further recruitment and a low pro-inflammatory profile. These data show that NadA is biologically active on monocytes and macrophages and is involved in eliciting tissue defence once the bacterium crosses the epithelial barrier, and that it promotes a Th2 response.

It will be understood that the invention has been described by way of example only and modifications may be made whilst remaining within the scope and spirit of the invention. One of skill in the art will recognize various alterations that may be practiced, based on the teachings herein, and such alterations are intended to be within the scope of some embodiments of the invention. All documents cited herein are incorporated by reference in their entirety for all purposes, to the same extent as if each reference were individually listed as being incorporated by reference.

### REFERENCES

### The Contents of which are Hereby Incorporated in Full

[1] Abbas et al. (1996) Nature 383(6603):787-93.
[2] Kalinski & Moser (2005) Nat Rev Immunol 5(3):251-260.
[3] Tettelin et al. (2000) Science 287:1809-1815.
[4] WO2004/113371
[5] Cornelis et al. (1998) Microbiol. Mol. Biol. Rev. 62:1315-1352.
[6] Chen et al. (1999) Infect. Immunol. 67:1310-1316.
[7] Tham et al. (1994) J. Bacteriol. 176(3):781-788.
[8] Ravdin et al. (1986) Infect. Immun. 53(1):1-5.
[9] Johnson (1991) Clin. Micro. Rev. 4(1):80-128.
[10] Jacobs et al. (1987) J. Bacteriol. 169(2):735-41.
[11] Dean-Nystron & Samuel (1994) Infect. Immun. 62(11):4789-4794.
[12] McGarey et al. (1994) Infect. Immun. 62(10):4594-4601.
[13] Burgess & McDonald (1992) Infect. Immun. 60(10):4254-9.
[14] U.S. Pat. No. 5,175,096
[15] U.S. Pat. No. 5,189,015
[16] U.S. Pat. No. 5,320,951
[17] U.S. Pat. No. 5,416,021
[18] U.S. Pat. No. 5,440,014
[19] Murphy et al. (1996) Prostate 29:371-380.
[20] O'Neill et al. (2004) Blood 104(8):2235-2246.
[21] Kugler et al. (2000) Nature Med. 6:332-336.
[22] Wen et al. (1998) Clin Cancer Res. 4:957-962.
[23] Hsu et al. (1996) Nature Med. 2:52-55.
[24] Nestle I. (1998) Nature Med. 4:328-332
[25] Chakraborty et al. (1998) Cancer Immunol Immunother. 47:58-64.
[26] Salgaller et al. (1998) Crit. Rev Immunol. 18:109-119.
[27] EP1470821
[28] US2005232932
[29] WO03/083089
[30] U.S. Pat. No. 4,680,338.
[31] U.S. Pat. No. 4,988,815.
[32] WO92/15582.
[33] Stanley (2002) Clin Exp Dermatol 27:571-577.
[34] Wu et al. (2004) Antiviral Res. 64(2):79-83.
[35] Vasilakos et al. (2000) Cell Immunol. 204(1):64-74.
[36] U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,656,938, 6,660,735, 6,660,747, 6,664,260, 6,664,264, 6,664,265, 6,667,312, 6,670,372, 6,677,347, 6,677,348, 6,677,349, 6,683,088, 6,703,402, 6,743,920, 6,800,624, 6,809,203, 6,888,000 and 6,924,293.
[37] Jones (2003) Curr Opin Investig Drugs 4:214-218.
[38] Kandimalla et al. (2003) Nucleic Acids Research 31:2393-2400.
[39] WO02/26757.
[40] WO99/62923.
[41] Krieg (2003) Nature Medicine 9:831-835.
[42] McCluskie et al. (2002) FEMS Immunology and Medical Microbiology 32:179-185.
[43] WO98/40100.
[44] U.S. Pat. No. 6,207,646.
[45] U.S. Pat. No. 6,239,116.
[46] U.S. Pat. No. 6,429,199.
[47] Kandimalla et al. (2003) Biochemical Society Transactions 31 (part 3):654-658.
[48] Blackwell et al. (2003) J Immunol 170:4061-4068.
[49] Krieg (2002) Trends Immunol 23:64-65.
[50] WO01/95935.
[51] Kandimalla et al. (2003) BBRC 306:948-953.
[52] Bhagat et al. (2003) BBRC 300:853-861.
[53] WO03/035836.
[54] WO01/22972.
[55] Myers et al. (1990) pages 145-156 of Cellular and molecular aspects of endotoxin reactions.
[56] Ulrich (2000) Chapter 16 (pages 273-282) of Vaccine Adjuvants: Preparation Methods and Research Protocols (Volume 42 of Methods in Molecular Medicine series). ISBN: 1-59259-083-7. Ed. O'Hagan.
[57] Johnson et al. (1999) J Med Chem 42:4640-9.
[58] Baldrick et al. (2002) Regulatory Toxicol Pharmacol 35:398-413.
[59] Thompson et al. (2005) J Leukoc Biol 78: ‘The low-toxicity versions of LPS, MPL® adjuvant and RC529, are efficient adjuvants for CD4+ T cells’.
[60] UK patent application GB-A-2220211.
[61] WO 94/21292.
[62] WO94/00153.
[63] WO95/17210.
[64] WO96/26741.
[65] WO93/19780.
[66] U.S. Pat. No. 5,108,910
[67] U.S. Pat. No. 5,391,485
[68] U.S. Pat. No. 6,054,297
[69] Overholser et al. (2000) Cancer 89:74.
[70] Yang et al. (2001) Crit Rev Oncol Hematol 38:17.
[71] Comanducci et al. 2002 J. Exp. Med. 195:1445-1454.
[72] Lanzavecchia & Sallusto (2004) Nat Immunol. 5(12):1201-2.
[73] Capecchi et al. (2005) Mol. Microbiol. 55:687-698.

1. A method of adjuvanting an immune response, comprising:
administering an effective amount of a composition comprising an adhesin.

2. The method as recited in claim 1 wherein said administering activates dendritic cells.

3. The method as recited in claim 1 wherein said adhesin comprises a soluble form of NadA.

4. The method of claim 3, wherein said soluble form of NadA is the fragment NadAΔ351-405.

5. The method of claim 1, wherein said composition further comprises an additional adjuvant and/or immunopotentiator.

6. The method of claim 5, wherein said additional adjuvant and/or immunopotentiator is selected from an immunostimulatory oligonucleotide, an oil-in-water emulsion, a mineral salt, an ISCOM, LPS or an imidazoquinoline compound.

7. The method of claim 1, wherein the composition further comprises an interleukin or an interferon.

8. The method of claim 7, wherein said interferon is IFN-γ.

9. A composition comprising an adhesin, an antigen and one or more of an immunostimulatory oligonucleotide, an oil-in-water emulsion, a mineral salt, an ISCOM, LPS or an imidazoquinoline compound.

10. The composition of claim 9, wherein said adhesin is a soluble form of NadA.

11. The composition of claim 10, wherein said soluble form of NadA is NadAΔ351-405.

12. The composition of claim 9, further comprising an interleukin or an interferon.

13. The composition of claim 12, wherein said interferon is IFN-γ.

14. Use of a composition according to any one of claims 9-13 for adjuvanting an immune response.

15. Use of a composition according to any one of claims 9-13 for activating and sensitising a dendritic cell.

16. The use of claim 15, wherein said dendritic cell is CD86⁻.
